(19) United States
(12) Patent Application Publication
(10) Pub. No.: US 2006/0188551 A1
(43) Pub. Date: Aug. 24, 2006

(54) Products comprising a sheet and a wax dispersion

(76) Inventors: Matthias Hauser, St Augustin (DE); Anne Sans, Koln (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/541,818

(22) PCT Filed: Jan. 8, 2004
(86) PCT No.: PCT/EP04/00143

§ 371 Date: Feb. 23, 2006

(30) Foreign Application Priority Data
Jan. 8, 2003 (EP) 030751010

(51) Int. Cl. A61K 9/70

(52) U.S. Cl. 424/443

(57) ABSTRACT

This invention concerns products for cleansing and other applications that comprise a sheet of absorbent material, in particular of non-woven material, such as a wipe, to which are applied a wax dispersion and an aqueous phase. The invention further concerns the manufacture and use of such products.

### FIELD OF THE INVENTION

This invention concerns products for cleansing and other applications, which products comprise a sheet of absorbent material, in particular of a non-woven material, such as a wipe, to which a wax dispersion has been applied. The invention further concerns the manufacture and use of such products.

### BACKGROUND OF THE INVENTION

Wipe products have become an important product category that has found a wide variety of applications for adults and babies. Examples include face or body cleansing wipes, wipes for skin treatment, and skin conditioning wipes. Over the last couple of decades so-called wet wipes have become successful as products particularly suited for these applications. These products typically are manufactured by impregnating sheets made of non-woven fabric with a suitable lotion.

Initially, wet wipe products were made of traditional non-woven materials based on paper making technology (pulp based products). These products were well accepted but deficient in softness of the fabric material. The introduction of the ‘spunlace’ non-woven technology resulted in products that, compared to traditional paper based products, were superior in terms of softness.

In addition to the above, lotions have been developed which offered skincare benefits in addition to the basic cleansing properties of the wipe. One approach was the introduction of lotions that were based on oil-in-water emulsions, which delivered useful properties such as superior mildness, moisturization, protection and skin smoothness, when compared to simple aqueous cleansing formulations. Another approach encompassed the incorporation of active skincare ingredients into aqueous formulations, thereby delivering useful properties such as soothing.

However, this type of products has several limitations. Firstly, only a small portion of the lotion (usually about 15%) is released from the wipes during use. Thus a large quantity of the relatively expensive lotion is not delivered to the skin providing no benefit to the consumer and is wasted when the product is discarded after use. This also prevents the use of expensive but more effective ingredients. Secondly, from a formulation point, there is an apparent contradiction in the optimization of cleansing performance and skincare benefits in one single lotion, since ingredients which are effective in cleansing usually are not compatible with efficient skin care agents.

Another important factor in cleansing is the fact that a number of soils are water-compatible and therefore more easily removed by water-based formulations, whereas others are lipid-compatible and therefore adequately removed by lipid or oil based formulations. A complete and effective removal of soils therefore requires the presence in or on a wipe of as well water and oil-based components. This in particular is the case in wipes used for babies and infants. In the latter instance wet wipes are used for cleansing the perianal region when changing diapers. Inadequate cleaning not only results in personal discomfort but also gives rise to diaper rash and other infection related phenomena. It has been shown that the most effective way of preventing diaper rash is to cleanse the skin thoroughly and to remove the micro-organisms that have been identified as causative. The source of these micro-organisms is often the fecal deposits that can remain on a baby's skin while wearing the diaper. Because fecal deposits consist of both water-soluble and oil-soluble matter, however, complete removal of fecal deposits from the diaper area requires both water-based and oil-based cleansing agents.

Other areas where there is room for improvement are the caring properties of the wipe products, their capability to deliver active ingredients to the skin, the skin-feel of the wipe product itself, good skin tolerance, as well as the after-use skin feel. Skin-feel often is greasy and ‘heavy’ (i.e. an unpleasant, not light skin-feel). All these so-called ‘sensorial’ properties of wipe products are important attributes leaving room for improvement. This is in particular the case for baby applications. Moreover there is need for products that are stable for sufficiently long periods of storage, have no unpleasant odor and are environmentally friendly in that they are readily decomposed into harmless decomposition products. The wipe products moreover should be easy to manufacture in that any compositions in or on the wipes can conveniently be applied thereto in efficient and cost-effective manufacturing procedures.

WO-96/14835 discloses dry tissues to which a water-in-lipid emulsion has been applied and WO-99/25923 concerns a process and an apparatus for selectively coating a wipe with a water-in-lipid emulsion. WO-99/01536 discloses wipes wherein the carrier comprises two regions of different basis weight being applied with an emulsion comprised of a solidified wax phase, a polar phase dispersed therein and an emulsifier. Other prior art in this field is WO-95/35411, WO-95/35412, WO-95/16824, WO-97/30216, DE-33 09 530 and the publication of R. E. Mathis in Nonwovens World 1999, pp. 59-65.

U.S. Pat. No. 4,987,632 discloses a substantially dry-to-the-touch wiping article for use in cleaning soiled surfaces wherein moisture barriers cover the surface of the sheet. WO-99/13861 and U.S. Pat. No. 6,153,208 disclose substantially dry personal cleansing articles wherein the substrate comprises multiple layers. U.S. Pat. No. 6,280,757 concerns cleansing articles that are dry comprising a substrate having apertures of certain size and frequency.

The products according to the present invention, which comprise a sheet that contains a wax dispersion, have been found to be effective cleansing tools and efficient applicators of skincare ingredients.

### SUMMARY OF THE INVENTION

This invention relates to products that comprise a porous or absorbent sheet whereto a wax dispersion has been applied. The wax dispersion is comprised of wax particles dispersed in an aqueous phase. The particles in the dispersion have a particle size, which is in the micrometer range.

The wax dispersion preferably is present at the surface or at the surface portion of one or of both sides of the sheet.

The present invention in particular is concerned with products that comprise a porous or absorbent sheet whereto a wax dispersion has been applied, wherein the wax dispersion comprises

(a) a wax phase comprising suitable wax components, said wax phase having a melting point or melting range which is above ambient temperature, or which in particular is equal or higher than 25° C., and

(b) an aqueous phase.

In specific embodiments, the wax phase mentioned under (a) is present in an amount of 1-75 w/w % and the aqueous phase is present in an amount of 25-99 w/w %, relative to the total weight of the dispersion. In further specific embodiments, the wax dispersion further contains a suitable emulsifier.

In a further aspect, this invention provides products as defined above wherein the wax phase in the dispersion is as described hereinafter in preferred embodiments I, II or III.

In particular, the present invention is concerned with products that comprise a porous or absorbent sheet whereto a wax dispersion has been applied wherein the wax dispersion comprises wax particles containing components selected from fats, waxes, fatty alcohols, fatty acids, dialkyl(ene) ethers, dialkyl(ene) carbonates, dicarbonic acids and hydroxyfatty alcohols, and any mixture of these components.

Further in particular, the present invention is concerned with products, as defined above, wherein the wax particles have an average particle size, which is in the range of 0.5 to 100 μm.

Preferred embodiments are products as specified herein wherein the wax dispersion has a high viscosity, e.g. a viscosity of 1,000 mPas or higher.

Further in particular, the present invention is concerned with products that comprise a porous or absorbent sheet whereto a wax dispersion has been applied, wherein the particles in the wax dispersion have a particle size which is in the range of 0.5 to 100 μm and wherein the wax dispersion comprises

(a) a wax phase having a melting point which is equal or higher than 25° C., that contains at least one oil or wax component selected from dialkyl(ene) ethers, dialkyl(ene) carbonates, dicarboxylic acids or hydroxyfatty alcohols or any mixture of these substances and at least contains one emulsifier, and

(b) an aqueous phase.

In a preferred embodiment the wax dispersions contain

(a) 1-75 w/w-% of a wax phase having a melting point higher than 25° C., that contains at least one oil or wax component selected from dialkyl(ene) ethers, dialkyl(ene) carbonates, dicarbonic acids or hydroxyfatty alcohols or any mixture of these substances and contains at least one emulsifier, and

(b) 25-99 w/w % of an aqueous phase, relative to the total weight of the complete composition.

Preferred wax dispersions contain 5-30 weight % of the above mentioned wax phase; particularly preferred wax dispersions contain 10-25 weight % of the wax phase relative to the total weight of the wax dispersion.

The products of the invention can be made by applying a wax dispersion to a sheet, in particular to a non-woven sheet, as finely dispersed compositions. They can be applied in a cold process, which is more convenient than when using waxes, which need to be applied as a melt and subsequently cooled. They can also be applied as concentrates with low water content, for example with a water content of only 25 weight %, or as highly diluted formulations, e.g. having a concentration of only 1 weight %.

Particular embodiments of this invention are products with a wax dispersion having wax particles comprising a combination of wax components selected from dialkyl(ene) ethers, dialkyl(ene) carbonates, dicarbonic acids or hydroxy fatty alcohols and any mixtures thereof. These particular wax dispersions have excellent sensorial properties, good caring properties and are characterized by their less ‘heavy’ and less greasy and dry skin feel.

The sheet in the products of the invention in particular is made of a non-woven material, more in particular a non-woven material made by the spunlace or the hydro-entanglement procedure.

In still a further aspect this invention relates to products that comprise a porous or absorbent sheet whereto a wax dispersion and an additional aqueous phase have been applied. The wax dispersion and additional aqueous phase may be applied to the sheet sequentially or simultaneously.

The present invention further concerns products that comprise a porous or absorbent sheet whereto a wax dispersion has been applied and which products have been dried.

In a further aspect this invention relates to products that comprise a porous or absorbent sheet whereto a wax dispersion and an additional aqueous phase have been applied and which products have been dried and are therefore dry or essentially dry. Dry refers to the situation where the water content is very low, i.e. lower than 1% and essentially dry means that the product contains limited amounts of water, e.g. less than 10% of the total weight of the product, preferably less than 8%, more preferably less than 5%, still more preferably less than 2%.

In one embodiment the invention concerns a product comprising a porous or absorbent sheet to which an additional aqueous phase has been applied, after which the product is dried, and to which subsequently a wax dispersion is applied. If desired, the thus obtained product may in turn be dried.

A particular embodiment concerns a product comprising a porous or absorbent sheet to which an additional aqueous phase has been applied and to which subsequently a wax dispersion is applied, whereafter the product is dried. Another embodiment concerns a product as defined herein comprising a porous or absorbent sheet to which a wax dispersion has been applied whereafter the product is dried and to which subsequently an additional aqueous phase is applied, and, if desired, whereafter the thus obtained product is dried.

In a preferred embodiment, the wax dispersion is present at the surface or at the surface portion of one or both sides of the sheet.

In another aspect there is provided a method of manufacturing a product as described herein, said method comprising applying to the sheet a wax dispersion and an additional aqueous phase, either subsequently or simultaneously. In a preferred method of manufacturing, said sheet is first coated with a wax dispersion and subsequently sprayed or impregnated with an additional aqueous phase.

In a further aspect there is provided a method of manufacturing a product as described herein, said method comprising applying to the sheet the specific wax dispersion having wax particles comprising a combination of wax components selected from dialkyl(ene) ethers, dialkyl(ene) carbonates, dicarbonic acids or hydroxy fatty alcohols and any mixtures thereof as described herein.

In still a further aspect there is provided the use of a product of the invention as a cleansing tool, in particular in personal care applications.

In another aspect the invention concerns the use of a product as described herein as an applicator of active substances.

In still another aspect the invention provides the use of a product as described herein as a combined cleanser and applicator of active substances.

### DETAILED DESCRIPTION OF THE INVENTION

The absorbent or porous sheet can take the form of a tissue, a wipe, towel, towelette, cloth, flannel and the like. The material may be flushable. As used herein, by ‘flushable’ is meant that the material will pass through at least 3 meters of waste pipe in two toilet flushes. The material may also be biodegradable.

Materials of which the sheet is made may be mono or multi-layered, woven or non-woven. They can be made of one or of several materials. Particularly preferred are non-woven materials that have a web structure of fibrous or filamentous nature, in which the fibres or filaments are distributed randomly or with a certain degree of orientation, the former being obtainable by air-laying or certain wet-laying processes, the latter in certain other wet-laying or in carding processes. The fibres or filaments can be natural, for example wood pulp, wool cotton, linen and the like, or synthetic, for example polyvinyls, polyesters, polyolefins, polyamides and the like.

Multi-layered sheet materials have two or more layers of the same or different materials, woven or non-woven, or layers obtained by different techniques. One embodiment is a material composed of three layers, e.g. polyethylene/pulp/polyethylene or viscose/polypropylene/viscose.

Typically the sheets have a weight per square meter in the range of 10 to 80 g/m², in particular of 20 to 70 g/m². Particular materials are of the non-woven type. Based on the raw material that has been used, two different types of products can be distinguished.

A first type of carriers is paper based. The raw materials for these carriers are made almost exclusively of cellulose-based fibres or filaments from plant cellular sources (pulp). These can be available from fresh wood-shavings or from recycled material (recycled paper). In a number of wipe applications, such as baby wipes, wipes for cleansing, wet paper towels and the like, high wet strength or firmness of the non-woven web is a desirable attribute. This can be achieved by the addition of binding materials. Examples of such materials are the so-called wet strength resins. In some cases additives are added in order to increase the softness of the end product.

In a second type the web is made mainly of staple fibre, e.g. based on cotton, wool, linen and the like.

Commercial products are made of cellulose fibres, synthetic fibres or mixtures of both. Polyester and polypropylene are known as suitable polymers for the preparation of synthetic fibres. Also in these products binders can be used to increase the firmness of the non-woven fabric.

Webs of increased strength can be obtained by using the so-called spunlace or hydro-entanglement technique. In this technique the individual fibres are twisted together so that an acceptable strength or firmness is obtained without using binding materials. The advantage of the latter technique is the excellent softness of the non-woven material.

Non-woven materials that are made of a mixture of pulp and staple fibre are also known. Such materials are available with binding materials, in particular those mentioned above, or without binding materials. In the latter instance the non-woven is preferably made by the spunlace or hydro-entanglement procedure.

In a preferred embodiment of the present invention, the sheet material is made of cellulose pulp with a small amount of binding material. The amount of binder in the sheet material is in the range of 5 to 20% (w/w). In a particularly preferred embodiment the non-woven sheet material is prepared by the water entanglement procedure and does not contain binding material.

The absorbing ability of the sheet material is of particular interest with regard to the applications envisaged by the present invention. During production the impregnating solution should be taken up quickly by the sheet. In certain embodiments of this invention the wipes will be packed in a stack of a plurality of wipes. In this instance the absorbing ability of the non-woven fabric should be such that a chromatographic effect (sinking down of the lotion) in the stack is avoided during storage. On the other hand it should be guaranteed that during the usage of the wipe the impregnating solution is delivered evenly to the skin and the active ingredients are released quantitatively.

The absorbing capacity of the sheet material is determined essentially by three different parameters: the surface weight of the sheet material, the nature of the raw material used in the manufacture and the manufacturing process used.

For the applications according to the invention the sheet materials typically have a surface weight from 10 g/m² to 80 g/m², preferably from 30 to 70 g/m² and more preferably from 40 to 60 g/m². The selection of the raw material of which the non-woven sheet material is made depends on the manufacturing procedure. Typically in the manufacture of non-woven sheets by the hydro-entanglement process, use is made of mixtures of cellulose fibres and synthetic fibres. The relative quantity of synthetic fibres in the non-woven fabric is from 0 to 100% and preferably is between 10 and 70%, more preferably in the range of 30 to 50% (all percentages being w/w).

According to this invention the sheet material is contacted with a wax dispersion as defined herein. In some embodiments the sheet is contacted with another phase, which may be a polymeric phase. In other embodiments more than one wax dispersion, of different composition, is applied to the sheet. In a variant of the latter embodiments, there may also be another phase, which may be a polymeric phase.

The wax dispersion may be applied to the whole sheet, i.e. continuously, or to parts of the sheet, i.e. discontinuously. Where more than one wax dispersion is applied, one of these may be applied continuously while another is applied discontinuously. The wax dispersion or dispersions can be applied at the surface or in the internal of the sheet. If applied at the surface, one or both wax dispersions can be present at one side or at both sides of the sheet, or one wax dispersion may be present at one side while the other wax dispersion is present at the other side of the sheet.

In the instance where one or more wax dispersions are applied discontinuously, they may be present in or at certain areas, in particular in or at one or more areas of the sheet. In that instance, the wax dispersion(s) may be present as one or more forms or shapes. For example they can be present as dots or spots, lines or stripes, as geometrical figures such as squares, rectangles, circles and the like, as symbols such as letters, text, logos, figures and the like, or as trademark signs, or any other such forms, or a combination thereof. The forms or shapes may be present over the entirety of the sheet or grouped in one or more areas, for example in a corner or in the centre area.

In one type of embodiments, one wax dispersion is applied on one or on both sides of the sheet in the form of stripes, dots or other forms covering the entire surface or only a part of the surface of the sheet. In further embodiments, one or more other wax dispersions can be added. This may be done in a second step, preferably after the application of the first wax dispersion, or simultaneously in a one step operation. In embodiments where multiple wax dispersions are applied, these preferably are applied sequentially to the sheet.

In other embodiments, there is provided a sheet whereto an additional aqueous phase and a wax dispersion have been applied sequentially or simultaneously. Said additional aqueous phase may be water or a water based formulation containing other ingredients, in particular the ingredients mentioned hereinafter. In such specific embodiments, the sheet is first contacted with an additional aqueous phase and subsequently with a wax dispersion. In other types of such embodiments, the sheet is contacted with a wax dispersion and subsequently with an additional aqueous phase.

The additional aqueous phase can also be applied at various stages in the production process of the end product, including during the production of the sheet material.

In the embodiments containing both an additional aqueous phase and a wax dispersion, the wax dispersion as well as the additional aqueous phase may be applied as described above, i.e. either the wax dispersion or the additional aqueous phase, or both, may be applied in an equal manner or differently, and may be applied continuously or discontinuously, at the surface or the interior of the sheet, at one or at both sides, in certain areas and/or as certain shapes.

In still a further aspect, this invention provides dry or essentially dry products, or products with limited water content. Such products can be obtained by applying a wax dispersion of low water content or by application of an appropriate drying step. A wax dispersion of low water content may contain less than 20% (w/w relative to the total weight of the wax dispersion) or less than 10% water. Multiple variants are possible:
a) The sheet is treated with a wax dispersion of the invention and subsequently dried.
b) The sheet is treated with an additional aqueous phase, treated with a wax dispersion and subsequently dried.
c) The sheet is treated with an additional aqueous phase, dried, treated with a wax dispersion, and optionally dried; the latter drying step can be discarded if a wax dispersion of low water content is applied.
d) The sheet is treated with a wax dispersion, an additional aqueous phase and dried.
e) The sheet is treated with a wax dispersion, dried, subsequently treated with an additional aqueous phase and the whole is optionally dried.

Preferably the product is dried after the additional aqueous phase has been applied. Since the additional aqueous phase can be applied at various points in the production of the end product, the drying step can also occur at various points in the production process. The product can also be dried after application of a wax dispersion.

In each drying step, drying may be performed to a different extend, i.e. partial drying or complete drying.

Further possible variants are those wherein multiple additional aqueous phases and/or dispersions are applied, followed by an appropriate drying step or multiple drying steps.

Also included is the possibility to apply multiple additional aqueous phases and multiple wax dispersions and to introduce several drying steps. In each step it is possible that the phase is applied to only a portion of the sheet, or to one side of the sheet, or both. Any combination of such applications of the phases is deemed within the ambit of the present invention.

Thus particular embodiments of the invention are products that are dry or essentially dry. Dry refers to the situation where the water content is very low, i.e. lower than 1%. As used herein, ‘essentially dry’ means that the product contains limited amounts of water, e.g. less than 10% of the total weight of the product, preferably less than 8%, more preferably less than 5%, still more preferably less than 2%. More generally ‘essentially dry’ means that after manufacture, no water or aqueous-based lotion is added to the sheet. As used herein a % is w/w to the total weight of the sheet with all materials incorporated therein or thereon.

The Wax Dispersion

The products according to the invention contain a wax dispersion, which is composed of an aqueous dispersion of wax particles. The latter in turn are composed of a wax material, herein referred to as the ‘wax phase’ and are dispersed in an aqueous medium, herein referred to as the ‘aqueous phase’. Hence the products of the invention carry a two phase system comprising a lipid and an aqueous phase.

As used herein, the term ‘wax’ refers to any natural or synthetic components or component mixtures having the following properties: a consistency which ranges from solid to semi-solid (creamy), from coarsely crystalline to microcrystalline or even amorphous, from transparent to opaque, having a relatively low melting point, being of relative low viscosity at temperatures slightly above their liquefying point, being oil soluble and having a melting point or range of above ambient temperature, in particular above 20° C. or above 25° C. Waxes can be composed of one or more components, synthetic as well as natural, and can in principle be composed of any oil soluble material having a waxy consistency, including mixtures thereof. Waxes may also contain amounts of components with a lower melting point or liquid components as long as the melting point or range of the total composition of the wax is above ambient temperature, in particular above 20° C. or above 25° C.

The wax phase in particular has a melting point or a melting range above room temperature, specifically above 25° C., for example in the range of 25 to 100° C., in particular in the range of 30 to 75° C., more in particular in the range of 35 to 50° C., or of 30 to 45° C., or of 35 to 45° C., preferably in the range of 32 to 40° C. or of 37 to 42° C. More preferably the melting temperature or melting range is above human body temperature. Most preferably the melting temperature or melting range approximates or is equal to human body temperature.

In many instances, upon usage of the products of this invention, the wax phase and the aqueous phase in the wax dispersion undergo some form of interaction, which may contribute to the beneficial properties of the products. The selection of the melting point or range of the wax phase may be critical for such interaction, but also the addition of certain components to either the wax phase or the aqueous phase or to both may contribute thereto. A number of such additional components are described hereinafter in more detail.

In some embodiments of this invention the wax phase in the wax dispersion may have a relatively higher melting point or range. The melting point or range may for example be higher than body temperature, e.g. higher than 40° C., or higher than 45° C. Upon application of such products, a more intense interaction between the two phases of the wax dispersion may be required, or the application of higher temperatures, to promote the interaction. In the latter instance the consumer may, for example, be required to contact the product first with hot water and then to apply it. In the former instance the aqueous phase may contain agents that promote a stronger interaction of the aqueous phase with the wax phase.

As used herein the term ‘melting range’ refers to a temperature range that starts from the temperature at which a substance or composition loses its solid consistency up to the temperature where it becomes completely liquid. A melting range is considered to be within a defined temperature range when it overlaps with that defined temperature range, or should be considered to be above a specified temperature when the range is above said temperature.

As used herein ‘ambient temperature’ or ‘room temperature’ refers to a temperature that is in the range of about 20 to about 25° C.

The wax phase can further contain small amounts of water, although the water content of the wax phase generally is low. In preferred embodiments of the invention, the wax phase contains less than 10 weight % of water, the water content in particular is lower than 6 weight %, more in particular the water content is lower than 3 weight-%. In preferred embodiments, the wax phase is water free. As used herein, ‘water free’ means that the phase is composed of materials of low water content to which no water has been added.

The wax phase in the wax dispersion can change to another state in that, during storage or upon usage by the consumer, the wax phase becomes semi-solid or liquid. This change of state may be induced by physical factors such as temperature or pressure but may also be induced by chemical factors, such as particular chemical components or by a photochemical reaction.

In particular embodiments, multiple wax dispersions, i.e. wax dispersions made of phases of different composition, may be applied to the sheet. For example one type of wax dispersion is applied to one side of the sheet while another type is applied to the other. Each of these wax dispersions may or may not contain one or more of the ingredients mentioned hereinafter, for example one or more ingredients selected from the active ingredients, the dyes, emulsifiers, and other ingredients mentioned hereinafter. In case of various dyes, multi-colored patterns may exist, for example, each wax dispersion may have a different color or may be uncolored.

In other particular embodiments, the wax dispersion may be composed of two or more different wax phases, i.e. contain wax particles composed of different wax phases. The size, composition and properties of the wax particles may differ and may be selected in function of the envisaged properties and uses of the end product.

The wax dispersions further contain an aqueous phase. The aqueous phase contains water and, optionally, other ingredients, e.g. ingredients that have a stabilizing effect on the wax dispersion. The aqueous phase can contain water-soluble actives water soluble, dispersible or gellifying polymers, humectants etc. Furthermore, the aqueous phase in the wax dispersions may contain any of the ingredients mentioned hereinafter as possible ingredients of the ‘additional aqueous phases’.

The wax dispersions may be concentrates, i.e. having low content of water, or may be diluted with water. Where the wax dispersions are concentrates their viscosity usually will be higher and such dispersions will have a more creamy or pasty consistency. Wax dispersions with higher viscosity may also be obtained by adding specific amounts of suitable thickeners. Wax dispersions of higher viscosity may be preferred in certain techniques for applying the wax dispersions to the sheet.

Preferred embodiments are products having a wax dispersion of high viscosity, e.g. the wax dispersion has a viscosity of 1000 mPas or more, or 5000 mPas or more, or 10,000 mPas or more, or 20,000 mPas or more. Although it is possible to use dispersions of very high viscosity, for practical reasons the viscosity of the wax dispersions by preference will not exceed 100,000 mPas. A particular useful range is between 10,000 and 100,000 mPas, preferably between 20,000 and 50,000 mPas.

The wax particles in the wax dispersions preferably are of sufficiently small size to ensure a stable dispersion. In general the average size of these particles is in the range of about 0.5 to about 100 μm. Of particular interest for the invention are wax dispersions wherein the average particle sizes are in the range from 1-50 μm and in particular from 5-30 μm.

The wax dispersion may be applied differently at each side of the sheet. For example one side may completely be covered while at the other side the wax dispersion is applied in a pattern, e.g. as stripes.

The Wax Phase

The wax phase comprises one or more components that are designated as ‘waxes’, which are materials of waxy consistency as outlined above. Waxes are materials that have a solid to semi-solid (or creamy) consistency, crystalline or not, being of relative low viscosity a little above their liquefying point. Waxes can be composed of one or more components, synthetic as well as natural, and can in principle be composed of or comprise any oil soluble material having a waxy consistency, including mixtures thereof.

Waxes that can be used may be synthetic or natural waxes, as well as other oil soluble materials that have a waxy consistency. Waxes also encompass materials such as fats of natural or synthetic origin (including mono-, di- and triglycerides and fatty acid alkylesters), and mixtures thereof with oils. They further comprise other waxy components such as higher alkanols (in particular fatty alcohols), higher alkanediols (in particular hydroxy fatty alcohols), higher carboxylic acids (in particular fatty acids, also including substituted fatty acids (in particular hydroxy substituted fatty acids, for example, 12-hydroxystearic acid), dialkyl(ene) ethers, dialkyl(ene) carbonates, dicarboxylic acids and derivatives (in particular the C₁₆-C₄₀-dialkylesters of dicarboxylic acids, e.g. the C₁₆-C₄₀-alkyl stearates, C₁₈-C₃₈-alkylhydroxystearyl stearates or C₂₀-C₄₀-alkyl erucates) and hydroxy fatty alcohols, and the like waxy components.

Any of these wax phases may contain homologous components that are liquid, as long as the total composition making up the wax phase has a waxy consistency. For example, waxy fats may contain oils, waxy fatty alcohols may contain liquid fatty alcohols, etc., in such amount that the total composition has a waxy consistency and in particular has the melting point or range specified above.

Wax Components

Natural waxes comprise waxes from vegetal origin, such as purcelline, shea butter, cocoa butter, Japan wax, esparto gras wax, cork wax, guaruma wax, rice shoot wax, ouricury wax, montan wax, sunflower wax, ceresine wax, sugar cane wax, carnauba wax, candelilla wax, lanolin, fruit-derived waxes, such as orange wax, lemon wax, grapefruit wax and bayberry wax, and the like, and of animal origin such as beeswax, woolwax, spermateci and bear fat, shellac wax, and the like. Natural waxes further comprise mineral waxes such as ceresine and ozokerite waxes. Preferred among these natural waxes are waxes of vegetal origin. Synthetic waxes comprise petroleum-based waxes such as paraffin, vaseline, petrolatum, micro wax. Further synthetic waxes are polyalkylene and polyethyleneglycol waxes, e.g. polyethylene wax; waxes based on chlorinated naphtalenes such as ‘Halowax’, synthetic hydrocarbon waxes, and the like, including mixtures thereof. Further waxes are chemically modified waxes, in particular hardened or hydrogenated waxes such as, for example, Montan-ester waxes, Sasol waxes and hydrogenated jojoba waxes.

Other wax components may be selected from the esters from aromatic carbonic acids, dicarboxylic acids, tricarboxylic acids and hydroxycarboxylic acids (e.g. 12-hydroxystearic acid) and saturated and/or unsaturated straight or branched alcohols, and further lactides of long-chain hydroxy carbonic acids. Examples are C₂₀-C₄₀-dialkylesters of dimeric acids, C₁₈-C₃₈-alkylhydroxystearoylstearates or C₂₀-C₄₀-Alkylerucates. Myristyl lactate is particularly attractive for use on wipes for skin care, because of its good affinity to the skin. As further useful components there can be mentioned silicone waxes.

Further wax components that can be used are C₃₀-C₅₀-alkyl bees wax; tri-C₁₆-C₄₀-alkyl citrates, e.g. tristearyl citrate, triisostearyl citrate, trilauryl citrate; ethyleneglycol di-fatty acid esters, in particular the ethylene glycol di-C₁₂-C₃₀-fatty acid esters, e.g. ethylene glycol dipalmitate, ethyleneglycol distearate, ethyleneglycol di(12-hydroxystearate).

In preferred embodiments, the wax phase comprises any of the components designated herein as ‘waxes’ and further components, which may be selected from C₁₂-C₂₄-fatty alcohols, mono-, di- or tri-esters of glycerine and C₁₂-C₂₄-fatty acids, mono- or diesters from ethylene glycol and C₁₂-C₂₄-fatty acids or any mixtures of these components.

The total amount of waxes in the wax phase in particular is at least 50% (e.g. from 50-99.9%, or from 50-99%), preferably at least 70% (e.g. from 70-99.9%, or from 70-99%), more preferably at least 90% (e.g. from 90-99.9%, or from 90-99%), w/w of the total amount of components making up the wax phase.

In a particular embodiment, the total amount of waxes in the wax dispersion is in the range of from 0.1 to 45%, preferably of from 5 to 30%, more preferably of from 10 to 25%, all % being w/w relative to the total weight of the wax dispersion.

In a particular aspect of this invention there are provided products as specified herein wherein the wax phase essentially consists of one or more waxes selected from the waxes mentioned herein, including mixtures thereof. The waxes can be present in various amounts, e.g. the amounts mentioned hereinabove or hereinafter.

Fats and Oils

The wax phase may comprise fats and also certain amounts of oils. When present, the amount of oils will always be such that the wax phase is sufficiently solid as to form a dispersion and in particular will have the melting points or melting ranges outlined above. Fats or oils which can be used in the wax phase comprise natural fats or oils, or natural fat or oil derivatives, in particular of vegetable origin. Examples are almond oil, soybean oil, sunflower oil, safflower oil, corn oil, kernel oil, canola oil, borage oil, evening primrose oil, grapeseed oil, wheat germ oil, avocado oil, jojoba oil, sesame oil, walnut oil, linseed oil, palm oil, olive oil, macadamia oil, castor oil, rapeseed oil, peanut oil, coconut oil, and turnip seed oil, and in particular the hardened derivatives thereof. The latter are obtained by hydrogenation of fats or oils. Preferred are hardened oils or fats from vegetal origin, e.g. hardened peanut oil, soybean oil, turnip seed oil, cotton seed oil, sunflower oil, kernel oil, linseed oil, almond oil, corn oil, olive oil, sesame oil, cocoa butter, shea butter and coconut oil, in particular hardened castor oil or palm oil.

The wax phase may further comprise fatty components isolated from these natural oils, i.e. pure triglycerides or mixtures thereof, or the latter components having been prepared by chemical synthesis. These so-called triglycerides (or triacyl glycerines) are esters of glycerines with fatty acids or fatty acid mixtures. The latter also include technical mixtures obtained by hydrolysis from oils or fats, or from fractions thereof, or by fractioning fatty acid mixtures after hydrolysis.

The fatty acids in said triglycerides may be saturated or unsaturated, straight or branch chained, substituted or unsubstituted. Preferred triglycerides are those glycerine esters derived from fatty acids, either saturated or unsaturated, having from 10 to 60, in particular from 12 to 36, more particularly from 12 to 24, preferably from 16 to 20 carbon atoms. Preferred such fatty acids are, for example, palmitic, palmic, oleic, lauric, myristic, stearic, hydroxystearic, behenic acid, or mixtures thereof. Amongst the triglycerides those derived from saturated fatty acids are of particular interest.

Preferred among the triglycerides are those that liquefy at temperatures which are in the range of 35-50° C., preferably from 35-45° C., more preferably from 37-42° C. Further preferred are triglycerides that contain saturated, unbranched and unsubstituted fatty acid residues. They can be mixed esters, i.e. tri-esters of glycerine with different fatty acids. Suitable triglycerides that can be incorporated are hardened castor oil, a triester from glycerine and a hydroxystearic acid, a product that is commercially available under the tradename Cutina HR™. Further such triglycerides are glycerine tristearate, also referred to as stearin, glycerine tribehenate, glycerine tripalmitate, glycerine trilaurate, glycerine trioleate, glycerine trimyristate.

The wax phase may also contain mono- or diglycerides, optionally in mixture with the fats and oils mentioned herein, in particular with triglycerides. The mono- or diglycerides for use in the wax phase are derived from saturated or unsaturated, linear or branch chained, substituted or unsubstituted fatty acids or fatty acid mixtures. Also in this instance the melting point or melting range of the wax phase preferably is as mentioned above, in particular is above ambient temperature, further in particular above 25° C., and more in particular is in the range of 32° C. to 40° C. Particular mono- or diglycerides are mono- or di-C₁₂-C₂₄ fatty acid glycerides, specifically mono- or di-C₁₆-C₂₀ fatty acid glycerides, for example glyceryl monostearate, glyceryl distearate. Mixtures of mono-, di- and, optionally, triglycerides can be derived from fractions of fatty acids. An example of such mixture for use as a component of the wax phase is a mixture of C₁₂-C₁₈ mono-, di- and triglycerides.

In particular embodiments of the invention, the wax phase contains mixtures of mono-. di- and triglycerides. Examples of such glyceride mixtures are mixtures of C₁₂-C₁₈-mono-, di- and triglycerides or stearic acid mono-, di- and triglycerides). Mixed esters as well as mixtures of mono-, di- and triglycerides are of particular interest because of their low propensity to crystallize and their capacity to improve the consistency of the formulation making up the wax phase.

The wax phase may also comprise alkyl esters of fatty acids, wherein the alkyl group in the latter has from 1 to 30 carbon atoms, preferably from 12 to 24 carbon atoms. The fatty acids in said alkyl esters in particular are C₁₂-C₃₀ fatty acids, more in particular C₁₂-C₂₀ fatty acids. The alkyl groups in said esters preferably are derived from fatty alcohols, as well as of mixtures thereof, which, for example, are obtained by high pressure hydrogenation of technical mixtures of the methyl esters derived from fats or oils.

Preferred are the alkyl esters of C₁₆-C₂₄ fatty acids, more preferably from C₁₆-C₁₈ fatty acids, and C₁-C₃₀ fatty alcohols, preferably C₈-C₂₄ fatty alcohols, more preferably C₁₂-C₂₀-fatty alcohols. Examples are the C₁₆-C₄₀-alkyl stearates, in particular the C₂₀-C₄₀-alkyl stearates.

Of particular interest in this regard are, e.g. stearyl stearate, palmityl stearate, stearyl behenate, cetyl stearate, cetyl behenate, cetyl palmitate, cetearyl behenate, behenyl behenate, stearyl heptanoate, stearyl octanoate, myristyl myristate, myristyl palmitate, myristyl stearate, myristyl isostearate, myristyl oleate, cetyl isostearate, cetyl oleate, stearyl isostearate, stearyl oleate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl isostearate, behenyl oleate, erucyl isostearate.

Of further interest are esters of linear C₆-C₂₂-fatty acids with branched alcohols, in particular 2-ethylhexanol, esters of branched C₆-C₂₂-fatty acids with linear alcohols, esters of C₁₈-C₃₈-alkylhydroxycarbonic acids with linear or branched C₆-C₂₂-fatty alcohols, esters of linear and/or branched fatty acids with poly-alcohols (e.g. propylene glycol, dimerdiol or trimertriol) and/or Guerbet alcohols, as well as esters of C₆-C₂₂-fatty alcohols and/or Guerbet alcohols with aromatic carbonic acids, in particular benzoic acid, esters of C₂-C₁₂-dicarbonic acids with linear or branched C₁-C₂₂-alcohols (e.g. dioctyl malate) or C₂-C₁₀-polyoles having 2 to 6 hydroxyl groups.

The wax phase may further contain other oily components, which are lipid components that are liquid at ambient temperature, in particular are liquid at 20° C. or at 25° C. These components in particular are any of the oily components described hereinafter in more detail.

The oily components can be any of the oils mentioned hereinabove as ‘oils and fats’, in particular any of these that are liquid at ambient temperature, in particular are liquid at 20° C. or 25° C. Of particular interest are the mono-, di- and triglycerides mentioned hereinabove that are liquid at ambient temperature, in particular are liquid at 25° C. The oily components can further be fatty acids and fatty alcohols, described hereinafter in the respectively sections, that are liquid at 25° C. The wax phase can contain various amounts of fats and oils. In certain embodiments, the fats and in particular the glycerides are present in an amount of up to 60% (e.g. from 0.1 to 60%), or in other embodiments up to 70% (e.g. from 0.1 to 70%), or up to 80% (e.g. from 0.1 to 80%) (w/w), relative to the total amount of the wax phase. In other embodiments, e.g. in those wherein the wax phase contains other components of waxy consistency, the glyceride mixture can be present in amounts in the range of 0.1-45 w/w %, preferably in amounts of 0.1-15 w/w % and most preferable 1-12 w/w % relative to the total weight of the wax dispersion.

In other embodiments, in particular those wherein the wax phase also contains dialkyl(ene)ethers or -carbonates, dicarboxylic acids or hydroxy fatty alcohols, the amount of said fatty ester glycerides are up to 50% (e.g. from 0.1 to 50%) and more preferably up to 40% (e.g. from 0.1 to 40%) (w/w), relative to the total quantity of the wax phase, or in the lower ranges mentioned in the preceding paragraph.

The total amount of fats or oils, or of mixtures of fats and oils and/or oily components in the wax phase in particular is at least 90% (e.g. from 0.1 to 90%), more in particular at least 70% (e.g. from 0.1 to 70%), or at least 50% (e.g. from 0.1 to 50%), w/w of the total amount of components making up the wax phase.

In a particular aspect of this invention there are provided products as specified herein wherein the wax phase essentially consists of fats or oils, or of mixtures of fats and oils and/or oily components, in particular those specified in this specification. The fats, oils and oily components can be present in various amounts, e.g. the amounts mentioned hereinabove or hereinafter.

In a further particular aspect of this invention there are provided products as specified herein wherein the wax phase consists essentially of one or more fatty acid glycerides selected from the mono-, di- or triesters from glycerine, or a mixture thereof. The glycerides can be present in various amounts, e.g. the amounts mentioned hereinabove or hereinafter.

Fatty Alcohols

The wax phase may also comprise fatty alcohols. Fatty alcohols that can be used are, for example, C₁₂-C₅₀-fatty alcohols, in particular the C₁₂-C₂₄-fatty alcohols that are derived from natural fats, oils or waxes such as, for example, myristyl alcohol, 1-pentadecanol, cetylalcohol, 1-heptadecanol, stearylalcohol, 1-nonadecanol, arachidylalcohol, 1-heneicosanol, behenylalcohol, brassidylalcohol, lignocerylalcohol, cerylalcohol or myricylalcohol, as well as Guerbet alcohols. Preferred for use in the present invention, are saturated, straight or branch chained fatty alcohols. However, also unsaturated, straight or branch chained alcohols can be used, optionally in admixture with saturated alcohols. Preferably the alcohols will be selected such that the melting point of the mixture is as referred to hereinabove and more in particular is in the range of 32 to 40° C.

Mixtures of fatty alcohols can evidently also be used, including fatty alcohol fractions obtained from the reduction of the corresponding fatty acid fractions derived from naturally occurring oils or fats such as, for example, almond oil, soybean oil, sunflower oil, safflower oil, corn oil, canola oil, borage oil, evening primrose oil, grapeseed oil, wheat germ oil, avocado oil, jojoba oil, sesame oil, walnut oil, linseed oil, palm oil, olive oil, castor oil, macadamia oil, rapeseed oil, peanut oil, coconut oil, and turnip seed oil.

Synthetic alcohols can also be used, such as, for example, the linear fatty alcohols of an even number of carbon atoms resulting from the Ziegler-synthesis (Alfole®) or the partially branched alcohols resulting from the Oxo synthesis (Dobanole®).

A preferred embodiment according to the present invention is that wherein the wax phase contains at least one fatty alcohol, more preferably at least one C₁₄-C₁₈-fatty alcohol. Also preferred is a wax phase with at least one C₁₆-C₁₈-Guerbet alcohol.

The total amount of fatty alcohols in the wax phase may vary and depends on the desired properties of the wax phase. In a number of instances it is desirable to have a relative higher quantity of fatty alcohols in the composition, in particular said alcohols will be present in an amount of 50% (e.g. from 50 to 99.9%, or from 50 to 90%), preferably at least 70% (e.g. from 70 to 99.9%, or from 70 to 90%), more preferably at least 90% (e.g. from 90 to 99.9%, or from 90 to 99%), (all w/w) of the total amount of components making up the wax phase. In other instances, relatively lower amounts are desired, the total amount of the fatty alcohols present in the wax phase is in the range of 0.1-50%, in particular of 1-40%, preferably of 1-30% (w/w), more preferably of 1-20% (w/w), still more preferably from 1-10% (w/w).

In one embodiment the total amount of fatty alcohols in the wax dispersion is in the range of from 0.1 to 45%, preferably from 1 to 25%, more preferably from 5 to 20%, w/w relative to the total weight of the wax dispersion.

In a particular aspect of this invention there are provided products as specified herein wherein the wax phase essentially consists of one or more fatty alcohols, in particular those specified in this patent specification, including mixtures thereof. The fatty alcohols can be present in various amounts, e.g. the amounts mentioned hereinabove or hereinafter.

Fatty Acids

The wax phase may also contain C₁₄-C₄₀-fatty acids, including mixtures thereof. Of particular interest are the C₁₆-C₃₀-fatty acids. These comprise, for example, myristic-, pentadecanoic-, palmitic-, margaric-, stearic-, nonadecanoic-, arachic-, behenic-, lignoceric-, cerotic-, melissic-, erucaic-, elaeostearic-, oleic-, lonolenic-, lauric acid as well as substituted fatty acids, e.g. hydroxy-substituted fatty acids such as, for example, 12-hydroxystearic acid, and the amides or monoethanolamides of these fatty acids.

The total amount of the C₁₄-C₄₀-fatty acids present in the wax phase, relative to the total weight amount of the wax phase, is in the range of 0.1-30% or the range of 1-30% (w/w), preferably of 1-20% (w/w), more preferably from 1-10% (w/w). In one embodiment the total amount of fatty acids in the wax dispersion is in the range of from 0.1 to 45%, preferably from 1 to 25%, more preferably from 5 to 20%, w/w relative to the total weight of the wax dispersion.

In a particular aspect of this invention there are provided products as specified herein wherein the wax phase essentially consists of one or more fatty acids, in particular those specified in this patent specification, including mixtures thereof. The fatty acids can be present in varying amounts, e.g. the amounts mentioned hereinabove or hereinafter.

Dialkyl(ene) Ethers or -Carbonates, Dicarboxylic Acids or Hydroxy Fatty Alcohols

The wax phase may also contain dialkyl(ene) ethers, dialkyl(ene) carbonates, dicarboxylic acids or hydroxy fatty alcohols, or mixtures thereof, which ethers, carbonates, acids or alcohols in particular are those described hereinafter. In a particular aspect of this invention there are provided products as specified herein wherein the wax phase essentially consists of one or more dialkyl(ene) ethers or -carbonates, dicarboxylic acids or hydroxy fatty alcohols, including mixtures thereof. The dialkyl(ene) ethers or -carbonates, dicarboxylic acids or hydroxy fatty alcohols can be present in various amounts, e.g. the amounts mentioned hereinabove or hereinafter.

The addition of dialkyl(ene) ethers or -carbonates, dicarboxylic acids or hydroxy fatty alcohols, including mixtures thereof, to the composition of the wax phase allows to optimize the properties of the wax particles, in particular their sensorial properties, i.e. the products, as well as the skin after the products have been applied, have a less greasier feel and also a less dry skin-feel, while having excellent skin caring properties.

Dialkyl(ene) Ethers

The dialkyl(ene) ethers are symmetric or asymmetric, straight or branch chained, saturated or unsaturated. Preferred are waxy, saturated C₁₆-C₃₀-dialkylethers, in particular C₁₆-C₂₄-dialkylethers. More preferred are C₁₆-C₂₀-dialkylethers, and particularly preferred are distearylethers and dibehenylethers. Dialkylethers of shorter chain length can also be used such as, for example, di-n-octylether, di-(2-ethylhexyl)-ether, laurylmethylether or octylbutylether, didodecylether, under the condition that the complete composition of the wax phase has the desired melting point.

These ethers can be obtained from the appropriate fatty alcohols in the presence of an acid catalyst following art-known procedures. Typical examples are the products that are obtained by the etherification of capron alcohol, capryl alcohol, 2-ethylhexyl alcohol, caprin alcohol, lauryl alcohol, myristyl alcohol, cetyl alcohol, palmoleyl alcohol, stearyl alcohol, isostearyl alcohol, elaidyl alcohol, petroselinyl alcohol, linolyl alcohol, linolenyl alcohol, oleyl alcohol, ricinus alcohol, elaeostearyl alcohol, arachidyl alcohol, gadoleyl alcohol, behenyl alcohol, erucyl alcohol and brassidyl alcohol, Guerbet alcohols, as well as mixtures thereof, which, for example, are obtained by high pressure hydrogenation of technical mixtures of the methyl esters derived from fats or oils. Of particular interest are the dialkyl(ene) ethers that are solid at 25° C.

Dialkyl(ene) Carbonates

The dialkyl(ene) carbonates are symmetric or asymmetric, straight or branch chained, saturated or unsaturated. Preferred dialkyl(ene) carbonates are waxy, linear or branch chained, saturated or unsaturated C₁₄-C₃₀-dialkyl(ene) carbonates. More preferred are C₁₆-C₂₂-dialkyl carbonates and amongst these the saturated linear C₁₆-C₂₂-dialkyl carbonates. Particularly preferred is distearyl carbonate. Also liquid dialkyl(ene) carbonates, such as, for example, dihexyl-, dioctyl-, di-(2-ethylhexyl)- or dioleylcarbonate, can be used, under the condition that the complete composition has the desired melting point.

These dialkyl(ene) carbonates can be obtained by re-esterification of dimethyl- or diethyl carbonates with the corresponding hydroxy compounds following art-known procedures. Typical examples of dialkyl(ene) carbonates are re-esterification products of dimethyl- and/or diethyl carbonate with capron alcohol, capryl alcohol, 2-ethylhexyl alcohol, caprinalcohol, lauryl alcohol, myristyl alcohol, cetyl alcohol, palmoleyl alcohol, stearyl alcohol, isostearyl alcohol, elaidyl alcohol, petroselinyl alcohol, linolyl alcohol, linolenyl alcohol, oleyl alcohol, ricinus alcohol, elaeostearyl alcohol, arachidyl alcohol, gadoleyl alcohol, behenyl alcohol, erucyl alcohol and brassidyl alcohol, Guerbet alcohols, as well as technical mixtures thereof, that can be obtained by hydratation of methyl esters derived from suitable oils or fats or oil or fat fractions. Of particular interest are those dialkyl(ene) carbonates that are solid at 25° C.

Dicarboxylic Acids

Dicarboxylic acids that can be used are, for example, C₉-C₃₄-dicarbonic acids. These comprise, for example, octadecanedioic acid, tetratridecanedioic acid, etc. Of particular interest are the azelainic acids, which are C₉-dicarboxylic acids.

Hydroxy Fatty Alcohols

The hydroxy fatty alcohols for use in the wax phase compositions are saturated or unsaturated, straight chain or branched. Preferred are C₁₂-C₃₀-hydroxy fatty alcohols, at which the position of the hydroxy-substituent depends upon the synthesis route and the starting materials that have been used. Included are, for example, 1,10-decanediol, 1,2-hexadecanediol, 12-hydroxystearyl alcohol or hydroxy-Guerbet alcohols. Preferred are those hydroxy fatty alcohols that are solid at 25° C., although liquid analogues can also be used, as long as the complete composition has the desired melting point. Particularly preferred is 12-hydroxystearyl alcohol.

The total amount of one or more of the dialkyl ethers, dialkyl carbonates, dicarbonic acids and the hydroxy alcohols present in the wax phase, relative to the total weight amount of the wax phase, is in the range of 0.1-30% or in the range of 1-30% (w/w), preferably of 1-20% (w/w), more preferably from 1-10% (w/w).

In one embodiment, the total amount of dialkyl ethers, dialkyl carbonates, dicarbonic acids and the hydroxy alcohols is in the range of from 0.1 to 30%, preferably from 0.5 to 20%, more preferably from 0.5 to 10%, w/w relative to the total weight of the wax dispersion.

Oil Components

The wax phase may also comprise one or more oil components. The latter are components or component mixtures that are non water-mixable and that are liquid at ambient temperature, in particular are liquid at 20° C. or at 25° C. These can be any liquid homologues of the components mentioned above as being waxes, e.g. liquid glycerides, hydrocarbons, silicone oils, ester oils and the like, but also liquid dialkyl(ene) ethers, dialkyl(ene) carbonates, dicarbonic acids and hydroxy fatty alcohols, and the like, as well as mixtures thereof. The total quantity of these oily components in the total composition of the wax phase preferably will be such that the wax phase is solid at room temperature, or that it has a melting point or range that is as specified hereinabove. In particular embodiments, the quantity of oil components in the wax phase is limited such that the melting point of the complete composition is above 25° C.

The oil components will typically be present in quantities of less than 40% (e.g. from 0.1 to 40%) (w/w), in particular less than 30 weight % (e.g. from 0.1 to 30%), in particular less than 20% (e.g. from 0.1 to 20%), or further in particular 0.1-15%, more in particular from 0.5-10% (w/w), preferably in quantities in the range of 2-10% or in quantities in the range of 0.5-5 w/w-%, relative to the total weight of the wax dispersion.

The oil components can be liquid mono-, di- or triglycerides, or mixtures thereof, in particular any of the above mentioned oils of the glyceride type, that are liquid that are of natural or synthetic origin. Of particular interest are glycerides derived from C₆-C₂₄-fatty acids, in particular C₆-C₁₈-fatty acids, more in particular C₈-C₁₈-fatty acids. An example of such glycerides are the coco glycerides, a mixture of predominantly di- and triglycerides with C₈-C₁₈-fatty acids.

Further oil components that can be added are the liquid Guerbet alcohols based on fatty alcohols having 6 to 18, in particular 8 to 10 carbon atoms.

Further oily components, which can be used in the wax phase, comprise silicone oils, mineral and paraffin oils and synthetic oils, either aliphatic or aromatic, as well as mixtures thereof. Examples of such oils are squalane, squalene, isohexadecane, isoeicosane, polydecene, and also members of the group of dialkylcyclohexanes.

The wax phase may further contain silicone oils such as, for example cyclic silicones, dialkyl- or alkylarylsiloxanes, e.g., cyclomethicone, dimethyl polysiloxane and methylphenyl polysiloxane, as well as the alkoxylated and quaternized analogs thereof. Appropriate non-volatile silicone oils are e.g. polyalkylsiloxanes, polyalkylarylsiloxanes and polyethersiloxane-copolymers.

Still further oil components are from the ester type, in particular any of the liquid esters described above in the section ‘oils and fats’. Of particular interest are liquid esters from linear, saturated or unsaturated C₆-C₂₂-fatty acids with linear or branched, saturated or unsaturated C₆-C₂₂-fatty alcohols respectively esters from branched C₆-C₁₃-carboxylic acids with linear or branched, saturated or unsaturated C₆-C₂₂-fatty alcohols. Examples of oil components of the ester type are the following: decyl oleate, coco caprylate/-caprate (available under the tradename Cetiolo SN), hexyl laurate, myristyl isostearate, myristyl oleate, cetyl isostearate, cetyl oleate, stearyl isostearate, isostearyl myristate, isostearyl palmitate, isostearyl stearate, isostearyl isostearate, isostearyl oleate, isostearyl oleate, oleyl myristate, oleyl isostearate, oleyl oleate, oleyl erucate, behenyl isostearate, erucyl isostearate, erucyl oleate. Further oil components are the esters from linear C₆-C₂₂-fatty acids with branched alcohols, in particular 2-ethylhexanol, esters from branched C₆-C₂₂-fatty acids with linear alcohols, esters from C₁₈-C₃₈-alkylhydroxycarboxylic acids with linear or branched C₆-C₂₂-fatty alcohols, esters from linear and/or branched fatty acids with multifunctional alcohols (e.g. propylene glycol, dimerdiol or trimertriol) and/or Guerbet alcohols, as well esters from C₆-C₂₂-fatty alcohols and/or Guerbet alcohols with aromatic carboxylic acids, in particular benzoeic acids, esters from C₂-C₁₂-dicarboxylic acids with linear or branched alcohols having 1 to 22 carbon atoms (e.g. dioctyl malates).

Still further oil components are any hydrocarbons that are liquid at 20° C. of natural or synthetic origin, that are either aliphatic or aromatic, such as, for example squalane, squalene, paraffin oils, isohexadecane, isoeicosane or polydecene as well as dialkylcyclohexane.

Further Components

The wax phase may contain further components, which may be of waxy nature or otherwise. The use of these further components allows to influence the sensorial properties, as well as the stability of the compositions, in particular after application to wipe material, and more in particular to stabilize the wax particles being in contact with the aqueous phase, and to adapt the desired release profile. Any further components may also be added to influence consistency, feel and appearance. These components will generally be insoluble or poorly soluble in water. Water-soluble components can also be included, typically in combination with a solubilizing or emulsifying agent and some water.

Examples of further components are superfatting agents, thickeners, polymers, active ingredients, powders or powdered ingredients, film forming agents, UV-filters, anti-oxidants, hydrotropic agents, preservatives, insect repellents, self-tanning agents, solubilizers, perfume oils, dyes, etc.

The further components can be present in the dispersions in a total amount ranging from 0.1-45 weight %, preferably from 5-30 weight % and more preferably from 10-25 weight %, relative to the total weight of the wax dispersion.

The wax phase may further contain consistency agents such as, for example, small amounts of alkali metal or alkaline earth metal as well as aluminium salts of C₁₂-C₂₄-fatty acids or C₁₂-C₂₄-hydroxyfatty acids, preferred being calcium-, magnesium-, aluminium- and in particular zinc stearates.

The wax phase may further contain disintegrating agents, which are agents that cause a disintegration of the physical integrity of the wax phase. Suitable disintegrating agents are agents that are subject to physical or chemical interactions, either by auto-interaction or by interaction between two agents. This results in a physical or chemical interaction with the wax phase. One type of disintegrating agents are those that release a gas e.g. by decomposition or by chemical reaction between two components. An example of a disintegrating agent is a solid mixture of abicarbonate and an acid such as sodium or potassium carbonate with a suitable organic acid, e.g. citric acid. Upon contact with water, e.g. upon contact with the aqueous phase, the disintegrating components will interact and liberate carbon dioxide which physically alters the wax phase. Such physical alteration may, for example, cause the wax particles to become homogeneously distributed on the sheet. This may positively influence any interaction between the aqueous phase and wax particles, which in turn may have a positive effect on the transfer to the skin of materials, e.g. active ingredients, in these phases. Another reason for using a disintegrating agent can be the altered visual aspect of the wax dispersion.

The wax phase may further contain components that are subject to a polymerization reaction either during or after application on the sheet material. Examples of such components are oligomers that during or after application on the sheet continue to polymerize with monomers or other oligomers. Other examples are agents that cause netting or co-polymerization. There can also be agents that inhibit polymerization for a specific period of time. Alternatively there can be agents that accelerate polymerization e.g. under influence of external factors such as heat, light or pressure.

The wax dispersion, may further contain dyes, either in the wax phase or in the aqueous phase or in both, that upon usage of the product change color due to a change of temperature or pressure. This will give the consumer a level of comfort and trust that the product delivers the wax phase to the skin, or in case of a wax phase containing active ingredients, that the latter are delivered onto the skin.

The wax phase may further contain dye-precursors, i.e. agents that become dyed upon influence of physical or chemical factors. In particular embodiments the wax phase may contain dye-precursors that react with certain agents that are present in the aqueous phase, so as to form a dye. Similarly, the dye-precursors may be present in the aqueous phase and become transferred into dyes upon interaction with certain chemicals incorporated into the wax phase.

The wax dispersion can also be formulated to or into beads or capsules. Particularly such beads are polymeric beads wherein the wax dispersion is entrapped in whatever form. The terms ‘beads’ or ‘polymeric beads’ are meant to comprise any form of discrete, free-flowing powders, beads or capsules which envelope, coat or contain a wax dispersion in a mono- or polymeric matrix or capsule. These terms are also meant to include porous beads or ‘microsponges’ and ‘microcapsules’, the latter being beads of smaller size. The beads may be coated with a suitable coating material that protects the interior of the bead or controls the release of the wax dispersion entrapped therein. Where the wax dispersion is viscous enough, e.g. by addition of sufficient amounts of thickener, the coating on the bead itself may contain a wax dispersion. In the latter instance, the coating is laid on an inert core or on a core containing wax dispersion and/or other ingredients.

Formulation of the wax dispersion in beads may be done for protecting the wax dispersion from external factors that may impact its integrity. However, it is mostly done for allowing controlled release of the wax dispersion.

The size of the capsules or beads is selected such that are sufficiently big to contain the dispersion, relatively larger sizes being preferred, e.g. in the mm or 0.1 mm range. This type of capsules or beads can be made of materials such as agar, glycolic acid polymers, and further components such as water, mineral oils, glycerine. They may contain further ingredients such as preservatives, dye(s), and the like.

Another type of beads or microcapsules are microsponges. These are materials sized up to about 300 μm (average diameter) having a large inner surface. These are obtained by polymerization of particular monomers. Wax dispersion material can be entrapped therein either during this polymerization process or afterwards.

The capsules or beads may optionally contain one or more suitable disintegrating agents, in particular those mentioned in this specification. Upon contact with the appropriate external factor, the disintegrating agents will cause the capsules to break open thus allowing release of the wax dispersion entrapped therein.

Release of the wax dispersion from the beads or capsules can be the result of the rupture of the coating or from the matrix. This may be the result of physical factors such as pressure, strain or by shearing forces upon use of the sheet product, e.g. by rubbing the product to the skin or to a surface. Release of the wax dispersion may be due to the semi-permeable or porous nature of the bead or its coating or due to external factors such as contact with liquid media that cause the wax dispersion to become extracted, or to dissolve or disintegrate the bead or its coating, or by temperature effects. The capsules can also be disintegrated under influence of certain chemicals, in particular by disintegrating agents incorporated into the capsules. Particular embodiments of the latter are capsules containing suitable amounts of bicarbonate and an organic acid which, upon contact with water, e.g. upon contact with the aqueous phase when using the sheet product, cause the capsules to disintegrate.

The beads or capsules can be made according to methodologies generally known in the art, for example by emulsion polymerisation.

The beads or capsules may be applied to any portion of the sheet but preferably they are concentrated at the surface or in the upper surface portion of the sheet. This allows maximal transfer of the wax dispersion to the skin or to the surface to which the product is applied.

Of particular interest for use in the invention are C₁₄-C₁₈-fatty alcohols as well as C₁₆-C₁₈-Guerbet alcohols.

The total amount of one or more of the C₁₂-C₅₀-fatty alcohols present in the wax phase, relative to the total weight amount of the wax phase, is in the range of 0.1-30% or in the range of 1-30% (w/w), preferably of 1-20% (w/w), more preferably from 1-10% (w/w).

In a specific embodiment II, the total amount of one or more of the C₁₂-C₅₀-fatty alcohols is in the range of from 0.1 to 45%, preferably from 1 to 25%, more preferably from 5 to 20%, w/w relative to the total weight of the wax dispersion.

### Preferred Embodiment III

In a preferred embodiment m the wax phase is a waxy composition comprising at least one oil or wax component selected from dialkyl(ene) ethers, dialkyl(ene) carbonates, dicarboxylic acids or hydroxy fatty alcohols or mixtures thereof, the total composition thereof having a waxy consistency. The waxy phase in this preferred embodiment may contain—depending on the required profile—further waxy lipids and oils. It is essential, however, that the melting point of the wax phase is above ambient temperature and in particular is above 20° or above 25°.

In a particularly preferred embodiment III the wax phase is a waxy composition comprising:

(a) at least one oil or wax component selected from dialkyl(ene) ethers, dialkyl(ene) carbonates, dicarboxylic acids or hydroxy fatty alcohols or a mixture thereof;

(b) an active ingredient.

Particular dialkyl(ene) ethers, dialkyl(ene) carbonates, dicarboxylic acids or hydroxy fatty alcohols for use in the wax phase of preferred embodiment III are those mentioned hereinabove.

The said preferred or particularly preferred wax composition preferably liquefies above 25° C. and/or has a water content of less than 10%, preferably less than 6%, more preferably less than 3%. In particular said preferred or further preferred waxy composition is water-free, and will be such that it is not decomposed by the aqueous phase. As used herein, water-free generally means that the phase is composed of materials of low water content to which no water has been added.

The wax phase having the preferred composition III can contain the same further ingredients as those described herein in relation to the wax phase in general, and may in particular contain further waxy lipid components or oils.

The wax phase having the preferred composition III can also contain liquid dialkyl(ene) ethers, dialkyl(ene) carbonates, dicarbonic acids or hydroxy fatty alcohols, however in such amounts that the melting point or range of the total composition of the wax phase does exceed 25° C., and more preferably is within the temperature ranges mentioned above.

The preferred wax compositions and in particular the preferred wax compositions I, II and III may contain further ingredients, in particular any ingredients having a waxy consistency, e.g. at concentrations of less than 20%.

In further preferred embodiments, the wax phase in the wax dispersions of the invention has the composition of preferred embodiments I, II or III and has a melting point which is in the range of 35-50° C., in particular of 35-45° C. and more in particular of 37-42° C. These are preferred conditions for allowing the wax phase, when applied to the sheet, remains finely dispersed and only upon application at body temperature to become liquid. Sheets that have been treated with such wax dispersions are particularly stable during storage and mixing of the phases is avoided.

In particularly preferred embodiments, the products of this invention have a wax dispersion that comprises:

(a) 1-50 weight % of a wax phase comprising:

(a1) 0.1-30 weight % of at least an oil or wax component selected from C₁₄-C₃₀-dialkyl ethers, C₁₄-C₃₀-dialkyl carbonates, C₄-C₃₄-dicarbonic acids or C₁₂-C₃₀-hydroxyfatty alcohols or any mixture thereof;

(a2) 0.1-10% (w/w) of at least one oil;

(a3) 0.1-10% (w/w) of at least one non-ionic emulsifier

(a4) 0.1-40% (w/w) of at least one further waxy lipid component; w/w relative to the total weight of the wax dispersion;

(b) 50-99% (w/w) of an aqueous phase; w/w relative to the total weight of the wax dispersion.

Polymers

In further embodiments of the invention, the wax dispersion contains at least one polymer, preferably in the aqueous phase. The presence of polymers helps to improve the ability to form the fine wax particles in the wax dispersions of the invention. The polymers are usually present in quantities ranging from 0.01-5 w/w-%, in particular from 0.05-3 and more in particular from 0.1-2 w/w-%, relative to the total weight of the wax dispersion.

Anionic, zwitterionic, amphoteric and nonionic polymers that can be used are, for example, vinylacetate/crotonic acid-copolymers, vinylpyrrolidon/vinylacrylate-copolymers, vinylacetate/butylmaleate/isobornylacrylate-copolymers, methylvinylether/maleic acid anhydride-copolymers and their esters, polyacrylic acids which are either non cross-linked or are cross-linked with polyoles, acrylamidopropyltrimethylammonium chloride/acrylate-copolymers, octylacrylamide/methylmethacrylate/tert.butylaminoethylmethacrylate/2-hydroxypropymethacrylate-copolymers, polyvinylpyrrolidone, vinylpyrrolidone/vinylacetate-copolymers, vinylpyrrolidone/dimethylaminoethylmethacrylate/vinyl caprolactam-terpolymers as well as optionally derivatized cellulose ethers and silicones.

Of particular interest are polysaccharides, in particular xanthan gum, guar-guar, agar-agar, alginate and tyloses, carboxymethylcellulose and hydroxyethylcellulose, polyacrylates (e.g. Carbopole® from Noveon or Synthalene® from 3V/Sigma), polyacrylamides, polyvinylalcohol and higher molecular polyethylene glycolmono- and -di-ester of fatty acids.

Appropriate cationic polymers are for example cationic cellulose derivatives, e.g. quaternized hydroxyethyl cellulose (commercialized under the trade name Polymer JR 400® by Amerchol), cationic starches, copolymers of diallylammonium salts and acrylamides, quaternized vinylpyrrolidone/vinylimidazole-polymers (for example Luviquat® of BASF), condensation products of polyglycols and amines, quaternized collagen polypeptides, such as, for example, lauryldimonium hydroxy-propyl hydrolyzed collagen (Lamequat®L/Grtinau), quaternized wheat polypeptides, polyethylene imines, cationic silicone polymers, e.g. amodimethicone, copolymers of adipinic acid and dimethylaminohydroxypropyldiethylenetriamine (Cartaretine®/Sandoz), copolymers of acryl acid with dimethyldiallylammonium-chloride (Merquat® 550/Chemviron), polyaminopolyamides, cationic chitine derivatives such as, for example, quaternized chitosans, optionally dispersed in microcrystalline form, condensation products derived from dihalogenallcylenes, such as, for example dibromobutane with bis-dialkylamines, e.g. bis-dimethylamino-1,3-propane, cationic guar-gum, such as, for example, Jaguar® CBS, Jaguar® C-17, Jaguar®C-16 from Celanese, quaternized ammonium salt-polymers, e.g. Mirapol® A-15, Mirapol® AD-1, Mirapol® AZ-1 from Miranol.

Preferred are water soluble or water-swellable polymers, in particular non-ionic and anionic polymers. Particularly preferred are polymers selected from the group of polyacrylates, polysaccharides polyacrylamides or any mixture of these polymers.

Emulsifiers

The stability of the fine-particulate wax dispersions of the invention is increased by adding suitable emulsifiers. These in particular are ionic and non-ionic emulsifiers, the latter being preferred. The emulsifiers can be of the W/O (preferably for use in the wax phase) or the O/W (preferably for use in the aqueous phase) type. The addition of an emulsifier allows the incorporation of hydrophilic components or agents, or also of small amounts of water into the wax phase, and vice versa of lipophilic components or agents into the aqueous phase.

Preferred are non-ionic emulsifiers, which typically have good skin compatibility and mildness and are environmently friendly. They moreover add to the stability of the fine-particulate dispersions. Of particular interest are combinations of W/O and O/W-emulsifiers which yield dispersions of increased stability and improved sensorial properties. The wax dispersions contain the emulsifier(s) in quantities which are in the range of 0.1 to 10 w/w %, in particular of 0.5 to 7 w/w % and more in particular of 1 to 5 w/w % relative to the total weight of the wax dispersion.

Non-Ionic Emulsifiers

The following are suitable non-ionic emulsifiers:

(1) Addition products of 2 to 50 moles of ethylene oxide and/or 1 to 20 moles propylene oxide to linear fatty alcohols having 8 to 40 C-atoms, to fatty acids with 12 to 40 C-atoms and to alkylphenols with 8 to 15 C-atoms in the alkyl rest.

(2) C₁₂₋₁₈-fatty acid mono- and -diesters of addition products of 1 to 50 moles of ethylene oxide to glycerine.

(3) Addition products of ethylene oxide to glycerine mono- and -diesters; and sorbitan mono- and -di-esters of saturated and unsaturated fatty acids with 6 to 22 C-atoms and their ethylene oxide addition products.

(4) Alkyl mono- and -oligoglycosides with 8 to 22 C-atoms in the alkyl rest and their ethoxylated analogues.

(5) Addition products of 7 to 60 moles of ethylene oxide to castor oil and/or hardened castor oil.

(6) Polyol- and in particular polyglycerine esters, such as e.g. polyol poly-12-hydroxystearate, polyglycerine polyricinoleate, polyglycerine diisostearate or polyglycerine dimerate; also applicable are mixtures of components of several of these substance classes.

(7) Addition products of 2 to 15 moles of ethylene oxide to castor oil and/or hardened castor oil.

(8) Partial esters derived from linear, branch chained, unsaturated or saturated C₆-₂₂-fatty acids, ricinoleic acid as well as 12-hydroxystearic acid and glycerine, polyglycerine, pentaerythrite, dipentaerythrite, sugar alcohols (e.g. sorbitol), alkylglucosides (e.g. methylglucoside, butylglucoside, laurylglucoside) as well as polyglucosides (e.g. cellulose), or mixed esters such as e.g. glyceryl stearate/citrate and glyceryl stearate/lactate.

(9) Polysiloxane-polyalkyl-polyether-copolymers and the respective derivatives thereof.

(10) Mixed esters from pentaerythrite, fatty acids, citric acid and fatty alcohols and/or mixed esters of fatty acids with 6 to 22 C-atoms with methylglucose and polyoles, respectively glycerine or polyglycerine.

(11) Polyalkylene glycols.

The addition products of ethylene oxide and/or of propylene oxide and fatty alcohols, fatty acids, alkylphenols, glycerine mono- and di-esters as well as sorbitan mono- and -diesters of fatty acids or of castor oil are known and commercially available products. Usually these are mixtures of homologues of which the average degree of alkoxylation corresponds to the ratio of starting quantities of ethylene oxide and/or propylene oxide and substrate, with which the addition reaction is conducted. Depending upon the degree of alkoxylation these products are either W/O- or O/W-emulsifiers. C₁₂₋₁₈-fatty acid mono- and -di-esters of addition products of ethylene oxide to glycerine are known as re-fatting agents in cosmetic applications.

Of particular interest are the non-ionic emulsifiers of group (4) in the above list. Non-ionic emulsifiers from the group of alkyloligoglucosides and in particular the C₈-C₂₂-alkylmono- and -oligoglucosides, are attractive due to their good skin-compatibility. Alkyl monoglucosides, having a sugar rest linked to an alkyl group, as well as alkyl oligomeric glucosides having a degree of oligomerisation of up to about 8, or mixtures thereof can conveniently be used. An example is a C₈-C₁₆-alkylglucoside with a degree of oligomerisation of between 1-2, which is commercially available under the tradename Plantacare®. Further suitable non-ionic emulsifiers are the acylglucamides.

Particular useful and mild emulsifiers are polyolpoly-12-hydroxystearates and mixtures thereof with other components, that are available under the tradename “Dehymuls® PGPH” (W/O-emulsifier) or “Eumulgin® VL 75” (1:1 w/w mixture with coco-glucosides, O/W-emulsifier) or Dehymuls® SBL (W/O-Emulsifier) from Cognis Deutschland GmbH. The polyol components of these emulsifiers can be derived from materials that have at least two and in particular 3 to 12 and more in particular 3 to 8 hydroxyl groups, and 2 to 12 carbon atoms.

Lipophilic W/O-emulsifiers in principle are emulsifiers with a HLB-value in the range of 1 to 8, that are described, for example, in Kirk-Othmer, “Encyclopedia of Chemical Technology”, 3^{rd} Ed., 1979, Vol. 8, p. 913. The HLB-value of ethoxylated products is calculated by the formula: HLB=(100−L): 5, wherein L is the percentage (in weight %) of lipophilic groups, i.e. of fatty alkyl- or fatty acyl groups in the ethylene oxide adducts.

Particularly attractive W/O-emulsifiers are the partial esters of polyoles, in particular of mono-, di-, tri-, or sesqui esters of fatty acids of polyoles, more in particular of C₃-₆-polyoles, such as, for example, glyceryl monoesters, partial esters of pentaerythrite or carbohydrate esters, in particular the sorbitan mono-, di-, tri- or sesqui fatty esters, in particular the sorbitan stearates, oleates, erucates, ricinoleates, hydroxystearates, isostearates, tartrates, citrates, and maleates. Also of interest are addition products of ethylene oxide to these sorbitan esters, in particular with 1 to 30, respectively 5 to 10 moles of ethylene oxide.

In case it is desirable to incorporate water-soluble active ingredients and/or small amounts of water into the wax phase it may be advantageous to add an emulsifier selected from the group of non-ionic O/W-emulsifiers (HLB-value: 8-18) and/or solubilizers. These can for example be the already mentioned ethylene oxide-adducts with a corresponding high degree of ethoxylation e.g. 10-20 ethylene oxide units in the case of O/W-emulsifiers and 20-40 ethylene oxide units for so-called solubilizers. Particularly attractive as O/W emulsifiers are Ceteareth-12 and PEG-20 stearate. Particularly attractive solubilizers are Eumulgin® HRE 40 (INCI: PEG-40 Hydrogenated Castor Oil), Eumulgin® HRE 60 (INCI: PEG-60 Hydrogenated Castor Oil), Eumulgin® L (INCI: PPG-1-PEG-9 Laurylglycol ether) and Eumulgin® SML 20 (INCI: Polysorbate-20).

Non-ionic emulsifiers of the group of alkyl oligoglycosides are particularly skin-compatible and therefore preferred as O/W-Emulsifiers. C₈-C₂₂-alkyl mono- and -oligoglycosides, their preparation and use have been described in the prior art. Oligoglycosides are meant to comprise oligomeric glycosides with a degree of oligomerisation of up to about 8. The degree of oligomerisation can also be a statistical average used for those products comprised of a specific range of oligoglycosides. An example is the product sold under the tradename Plantacare®, which has a C₈-C₁₆-alkyl group glycosidically bound to an oligoglucoside rest, having an average degree of oligomerisation between 1 and 2.

Other non-ionic emulsifiers are the acyl glucamides. Preferred is the product sold under the tradename Emulgade® PL 68/50 (Cognis Deutschland GmbH) which is a 1:1-mixture of alkyl polyglucosides and fatty alcohols, and a mixture of lauryl glucoside, polyglyceryl-2-dipolyhydroxystearate, glycerine and water, sold under the trade name Eumulgin® VL 75.

Additional Ingredients for Either One or Both Phases in the Wax Dispersions

The wax dispersions of the invention may contain further ingredients that may be present in the wax phase or in the aqueous phase that carries the dispersed particles or in both. Examples are active ingredients, moisturizers, anti-perspirants, UV-absorbing agents, etc.

Active Ingredients

The wax dispersion may contain active ingredients for application to the skin. The wax phase in the wax dispersions preferably contains oil-soluble or hydrophobic active agents, while the aqueous phase preferably contains water-soluble or hydrophilic active agents. However by using suitable emulsifiers oil-soluble or lipophilic active ingredients can be incorporated into the aqueous phase and vice versa, water-soluble or hydrophilic agents can be incorporated in the wax phase of the wax dispersions.

The active ingredients, which may be lipophilic or hydrophilic, can be mixed with or incorporated into suitable carriers. These comprise any skin-acceptable inert materials that are known for formulating active ingredients. The carriers can be finely or more coarsely divided powders, or even granulates. They can comprise starches, sugars, binders, lubricants, diluents, fillers, disintegrating agents, granulating agents and the like components. The nature of the carrier materials will depend on the active ingredient that is formulated therein and on the type of formulation that is desired.

Particular carriers for incorporating active ingredients are beads wherein the active ingredient is entrapped in some form. The terms ‘beads’ or ‘polymeric beads’ are meant to comprise any form of discrete, free-flowing powders, beads or capsules which envelope, coat or contain an active ingredient in a mono- or polymeric matrix or capsule. These terms are also meant to include porous beads or ‘microsponges’ and ‘microcapsules’, the latter being beads of smaller size. The beads may be coated with a suitable coating material that protects the interior of the bead or controls the release of the active ingredient entrapped therein. The coating on the bead itself may contain the active ingredient in which case the coating is layed on an inert core.

Formulating an active ingredient in beads can be for protecting the active from environmental factors but is mostly done for allowing controlled release of the active.

A particular type of beads are small beads or capsules, having an average diameter which is in the micrometer range, although the average diameter can be as small as even 200 nm.

This type of capsules can be liposome-based, made for example of phospholipids such as lecithin, phosphatidyl ethanolamine, phosphatidyl serine, phosphatidic acid and the like. This type of capsules also can be made of starch, cellulose, porous gelatin and the like.

The capsules or beads can also be relatively larger, having average sizes in the mm or 0.1 mm range. This type of capsules or beads can be made of materials such as agar, glycolic acid polymers, and further components such as water, mineral oils, glycerin. They may contain further ingredients such as preservatives, dye(s), and the like.

Another type of beads or microcapsules are microsponges. These are materials sized from about 5 to about 300 μm (average diameter) having a large inner surface. These are obtained by polymerization of particular monomers. An active ingredient can be entrapped therein either during this polymerization process or afterwards. Microsponge-based carriers may be used to protect the active ingredient entrapped therein or for controlled release purposes.

The capsules may optionally contain one or more suitable disintegrating agents, in particular those mentioned in this specification. Upon contact with the appropriate external factor, the disintegrating agents will cause the capsules to break open thus allowing release of the active ingredient entrapped therein.

The capsules can be incorporated into the lipid or the aqueous phase or into both. They can also be applied to the sheet prior to the introduction of the lipid and aqueous phase. They can even be introduced during the manufacturing process of the sheet itself.

Release of the active from the beads or capsules can be the result of the rupture of the coating or the matrix. This may be the result of physical factors such as pressure, strain or by shearing forces upon use of the sheet product, e.g. by rubbing the product to the skin or to a surface. Release of the active ingredient may be due to the semi-permeable or porous nature of the bead or its coating or due to external factors such as contact with liquid media that cause the active ingredient to become extracted, or to dissolve or disintegrate the bead or its coating, or by temperature effects. The capsules can also be disintegrated under influence of certain chemicals, in particular by disintegrating agents incorporated into the capsules. Particular embodiments of the latter are capsules containing suitable amounts of bicarbonate and an organic acid which, upon contact with water, e.g. upon contact with the aqueous phase when using the sheet product, cause the capsules to disintegrate.

The beads or capsules can be made according to methodologies generally known in the art, for example by emulsion polymerization.

The beads or capsules may be applied to any portion of the sheet but preferably they are concentrated at the surface or in the upper surface portion of the sheet. This allows maximal transfer of the active ingredient to the skin or to the surface to which the product is applied.

The beads or capsules can be applied to the sheet in dry form by dusting, sifting, spraying and the like methods. They can also be printed or roll-coated in the form of a suitable liquid or paste. They can also be mixed with a suitable liquid, which can be a solvent that is inert towards the beads, or water, or an aqueous phase, and sprayed onto the sheet.

Examples of active agents which may be hydrophobic or hydrophilic for use in the products of the invention comprise compounds that have a cosmetic or therapeutic effect on the skin, hair, or nails, e.g., lightening agents, darkening agents such as self-tanning agents, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, firming agents, anti-callous agents, and agents for hair, nail, and/or skin conditioning.

Examples of active ingredients are hydroxy acids, benzoyl peroxide, sulfur resorcinol, ascorbic acid, D-panthenol, hydroquinone, octyl methoxycinnimate, titanium dioxide, octyl salicylate, homosalate, avobenzone, polyphenolics, carotenoids, free radical scavengers, spin traps, retinoids such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, peptides such as those disclosed in WO-00/15188, lipoic acid, amino acids such a proline and tyrosine, vitamins, lactobionic acid, acetyl-coenzyme A, niacin, riboflavin, thiamin, ribose, electron transporters such as NADH and FADH2, and other botanical extracts such as aloe vera and soy, and derivatives and mixtures thereof. Of particular interest are active agents that can be used for treating skin that shows inflammatory reactions, that is irritated, red or damaged. Examples of such agents are zinc compounds or sulphur.

The cosmetically active agent will typically be present in the formulation of the invention in an amount of from about 0.001% to about 10% by weight of the dispersion, e.g., about 0.01% to about 10% such as about 0.1% to about 5%.

Examples of vitamins include, but are not limited to, vitamin A, vitamin Bs such as vitamin B3, vitamin B5, and vitamin B12, vitamin C, vitamin K, and vitamin E and derivatives thereof.

Typical examples of anti-microbial agents are those active against gram-positive bacteria such as 2,4,4′-trichloro-2′-hydroxydiphenyl ether, chlorohexidine (1,6-di-(4-chlorophenyl-biguanido)hexan) or TCC (3,4,4′-trichlorocarbanilide). Furthermore many odorants and etheric oils have anti-microbial activity. Also glycerine monolaurate, glycerine stearate, glycerine oleate as well as glycerine dioleate have been found to possess anti-microbial activity and are particularly attractive for use in products that are applied on babies because of their mildness and lack of side effects. The quantity of anti-microbial agents can vary but usually is in the range of about 0.1 to 2% (w/w)—relative to the total amount of the wax dispersion.

Further Additional Ingredients

The wax dispersions can contain further ingredients such as moisturizers, refattening agents, thickeners, powders, biogenic actives, deodorants, film formers, UV sunscreen filters, anti-oxidants, hydrotropes, preservatives, insect repellents, self tanners, solubilizers, perfumes, dyes, pigments, and the like.

Preferred anti-oxidants are carotinoids, carotines (e.g. α-carotine, β-carotine, lycopine) and derivatives thereof, α-hydroxyacids (e.g. citric acid, lactic acid, malic acid), EDTA, vitamin C and derivatives (e.g. ascorbyl palmitate, Mg-ascorbyl phosphate, ascorbyl acetate), tocopherole and derivatives (e.g. vitamin-E-acetate), butylhydroxy toluol and butylhydroxy anisol.

Biogenic active ingredients are for example tocopherol, tocopherol acetate, tocopherol palmitate, ascorbic acid, desoxyribonucleinic acid, retinol, bisabolol, allantoin, phytantriol, panthenol, α-hydroxycarbonic acids, amino acids, ceramides, pseudoceramides, essential oils, extracts and vitamin complexes.

Moisturizers

The wax dispersions, either in the wax phase or in the aqueous phase, may further contain one or more moisturizers. These are added to improve the sensorial properties as well as to regulate skin hydration. These agents additionally can improve the penetration of the composition in or into the sheet.

Moisturizers typically are present in quantities of 1-20% (w/w), preferably of 5-15% (w/w), and more preferably 5-10% (w/w) relative to the total amount of the wax dispersion.

Suitable moisturizers are a.o. amino acids, pyrrolidone carbonic acid, lactic acid and its salts, lactitol, urea and urea derivatives, ureic acid, glucosamine, creatinine, hydrolysis products of collagen, chitosan or chitosan salts/-derivatives, and in particular polyols and polyol derivatives (e.g. ethylene glycol, propylene glycol, butylene glycol, pentylene glycol, hexylene glycol, erythrite, 1,2,6-hexanetriol, polyethylene glycols such as PEG-4, PEG-6, PEG-7, PEG-8, PEG-9, PEG-10, PEG-12, PEG-14, PEG-16, PEG-18, PEG-20, PEG-135, PEG 150), sugar and sugar derivatives (a.o. fructose, glucose, maltose, maltitol, mannite, inosite, sorbite, sorbityl silandiol, sucrose, trehalose, xylose, xylit, glucuronic acid and its salts), ethoxylated sorbitol (Sorbeth-6, Sorbeth-20, Sorbeth-30, Sorbeth-40), honey and hydrogenated honey, hydrogenated starch hydrolysates, as well as mixtures of hydrogenated wheat protein, hydrolyzed milk protein, lecithin, pythantriol, hyaluronic acid and salts thereof, and PEG-20-acetate copolymers. Particularly preferred moisturizers are glycerine, diglycerine and triglycerine.

The products according to this invention can be used as anti-perspirants or deodorants, in particular as wipes or tissues for use in these applications. In products for these applications either one or both phases contain actives that have deodorizing and/or anti-perspirant properties. Actives that can be used to this purpose are anti-perspirant agents such as, for example, aluminium chlorohydrates, aluminium-zirconium-chlorohydrate as well as zinc salts.

The products according to the invention can also be used in sunscreen applications and in that instance take the form of sunscreen wipes. In these products the wax particles and/or aqueous phase contains one or more sunscreen filters which are for example organic substances that are capable of absorbing ultraviolet radiation and to set free the absorbed energy as longer-wave radiation, e.g. as thermic energy. UVB-filters can be oil or water-soluble.

Apart from the above-mentioned soluble substances, there can also be used insoluble sunscreen pigments, namely finely dispersed metal oxides or metal salts.

Preparation of the Wax Dispersions

The wax dispersions can be obtained by preparing a pre-emulsion containing the wax components in an aqueous phase at a temperature above the melting point or melting range of the wax phase (usually above 40-90° C.) and introducing this pre-emulsion while being at the previously mentioned temperature into a cold aqueous phase (usually at a temperature in the range of 1-30° C.) that contains the polymeric components. The temperature of the cold aqueous phase preferably is lower or equal to 25° C., particularly preferred is a temperature of 5-25° C. During the addition of the ‘warmer’ pre-emulsion the temperature of this cold aqueous phase should be kept such that it does not reach the melting point or range of the wax phase, and consequently cooling may be required. The wax components can be selected among any of the above-mentioned wax components.

The polymeric components preferably are water-soluble or water swellable. By using polymers very fine particles and stable wax dispersions are obtained, which even do not separate over long periods of time. Preferably, the pre-emulsion contains a polymer as well. Appropriate polymers have been described before. In a preferred execution of the process the polymer is selected from the group of polyacrylates, polysaccharides, polyacrylamides or any mixture of these polymers.

The incorporation of the hot preemulsion into the cold water phase can be done by applying known methods. To obtain finely dispersed particles it is preferred to homogenize the preemulsion at least once before adding it to the water phase. In a further preferred embodiment the preemulsion is cooled using a heat exchanger before addition to the water phase, preferred to temperatures below 50° C. Further preferred is to spray the preemulsion under pressure through a nozzle into the water phase. The set up for the exact pressure conditions is dependent on the used equipment.

In a further preferred execution of the process, the aqueous phase contains an oil or wax component selected from the group of dialkyl(ene) ethers, dialkyl(ene) carbonates, dicarboxylic acids or hydroxyfatty alcohols or any mixture thereof and at least one emulsifier. Particular such components have been mentioned herein.

Preferably the quantities of the starting materials are selected such that wax dispersions are obtained that comprise (a) 1-75 w/w-% of a wax phase and (b) 25-99 w/w-% of a water phase, w/w relative to the total weight of the wax dispersion.

Application of the Wax Dispersion

The wax dispersion may be applied to the sheet in various ways. Preferably the wax dispersion is applied at the surface or at the surface portion of the sheet, on one or on both sides. The wax dispersion can be applied evenly or non-evenly to the sheet, non-evenly meaning that the distribution of the amount of the wax dispersion varies over the area of the sheet, i.e. some areas of the sheet can have greater or lesser amounts of the wax dispersion. Preferably the wax dispersion is evenly applied to the area of the sheet. The wax dispersion can be applied discontinuously or continuously to one or both sides of the sheet, or it may even be applied as a complete covering of one or both surfaces of the fabric.

The wax dispersion preferably is applied in a discontinuous pattern, to one or both sides of the sheet. To this purpose the wax dispersion is applied in a predetermined, controlled manner to specific areas of the sheet. A discontinuous pattern is one in which the wax dispersion has been applied to distinct regions separated by regions of the sheet which are free of the wax dispersion. The wax dispersion in that instance is applied to defined parts or regions of the sheet, which may take a variety of forms. The wax dispersion may in particular be applied as described above more generally for the application of both phases. Particular forms in which the wax dispersion may be applied are, e.g. stripes, dots or spots, geometric configurations, either of regular or irregular shape, for example circles, ellipses, squares, rectangles and the like, logos, text, letters or any other non-continuous pattern, including the patterns described hereinabove more generally for the application of the lipid and aqueous phase.

Discontinuous patterns also comprise essentially networks of larger patterns of the wax dispersion. In a preferred embodiment, the wax dispersion is present as discrete stripes, which can be disposed discontinuously, i.e. interrupted, or preferably continuous over the whole surface of the wipe. The stripes may also form a pattern of discrete segments which collectively comprise a stripe or they may have a repetitive pattern such as a sinusoidal shape or wave-like and the like pattern. If waving stripes are selected, preferably the stripes are in phase, so that parallelism is maintained and each stripe remains equally spaced from the adjacent stripes. The stripes are preferably oriented in the machine direction, for ease of manufacture.

In another embodiment more than one wax dispersion may be applied to one or both sides of the sheet. For example one wax dispersion may be applied on the entire surface or part of the surface of one side of the sheet, whereas another wax dispersion is applied on the entire other side or only partly, either with the same or another pattern than the other wax dispersion. In particular such embodiments are those having two different wax dispersions on the same side e.g. in parallel stripes or other patterns with the same or different colors.

In a particular embodiment, not more than half of the surface of the sheet, either on one side or, which is preferred, on both sides is carrying or covered by the wax dispersion. In a preferred embodiment, the wax dispersion is present at the surface on both sides, covering not more than 50% of the sheet's surface, in particular covering not more than 35% or not more than 25% of the surface. In a particularly preferred embodiment, the wax dispersion is present as stripes, in particular as parallel stripes running in parallel with the side of the sheet, covering not more than half or, more in particular 25% of the surface. In another particularly preferred embodiment, the wax dispersion is present as dots, equally spread over the entire surface of the sheet, covering not more than 50% of the surface.

There can be embodiments with more or less regularly shaped dots, other embodiments have circle-shaped dots, others have ellipsoids, while still others have mixed patterns, e.g. combinations of circles and ellipsoids, of regularly shaped dots and circles and the like.

In case of stripes, the width thereof preferably is between 1 to 10 mm, more preferably between 3 to 7 mm. In case of dots, round shapes are preferred, e.g. circles or ellipsoids, with an average diameter between 1 to 10 mm, more preferably between 3 to 7 mm. There can be stripes with different widths on one product, and there can be dots of different size on one product. An example of an embodiment of the latter is a sheet with circles of a certain size and ellipses of a different size, or of circles with different sizes.

The wax dispersion may be colorless or colored, i.e. mono- or multi-colored. Multi-colored patterns are obtained by applying several wax dispersions that have been dyed differently. A colored wax dispersion will alert the user of the fact that the sheet is covered by a special material that contains an active ingredient or it may also make the product aesthetically attractive.

In another embodiment the sheet itself is colored, either at both sides or at one side, over the complete surface or only at parts. If the color is present only at parts of the sheet it preferably will take the shapes and forms described in connection with the patterns that the wax dispersion might take. In another embodiment only the space between the surface portions at which the wax dispersion is applied is colored thus leaving the areas of the wax dispersion uncolored. In this way, the patterns of the wax dispersion will appear as uncolored patterns.

A preferred pattern for coloring the sheet is in stripes, in particular stripes oriented in the machine direction. Examples of such embodiments are those wherein the colored stripes or the area between the colored stripes are covered with wax dispersion. In the former instance the wax dispersion stripes are colored, in the latter they are uncolored.

The wax dispersion, which itself can be colored or uncolored, may be applied to the colored sheet in a number of different ways.

In case of sheets having a completely colored surface, the wax dispersion can be applied over the whole surface thus resulting in a different or altered color, e.g. a more pale color where the wax dispersion is white or opaque. The wax dispersion can also be applied in certain patterns, thus resulting in multicolored products or where the wax dispersion is white or opaque in products with mono-colored patterns. Also in this instance, the preferred pattern is in stripes.

In still a further embodiment, the sheet is colored in certain patterns and the wax dispersion is applied on these patterns or part of these patterns. Also in this instance the wax dispersion may be colored or uncolored, i.e. white, opaque or transparent. In case the wax dispersion is white or opaque its thickness may be selected such that the color of the underlying section of the sheet is visible thus giving the consumer the impression that a wax dispersion containing a particular ingredient is present.

The wax dispersion is typically applied in an amount of about 3 to about 200 g/m², preferably of about 3 to about 40 g/m², more preferably from about 10 to about 20 g/m², either on one side or, preferably, on both sides of the sheet. Or, alternatively, the wax dispersion is applied in an amount of about 0.06 g to 1.50 g per gram of substrate, preferably from about 0.20 g to 0.70 g per gram of dry substrate.

The wax dispersion can be applied to the sheet by any method that can be used to contact or impregnate a liquid or semi-liquid material to or in a sheet. The wax dispersion may be applied by bathing the sheet into the wax dispersion. Where the latter is semi-solid, it can be made more liquid by dilution with water or an aqueous phase, which may be evaporated afterwards.

The wax dispersion can also be applied by any method that allows coating of the lipid material onto the surface of the sheet. As used herein the term ‘coating’ refers to printing, covering, overlaying, finishing, spraying, extruding, laminating or any other method of applying the phase to the surface of the sheet.

A particular coating technique is extrusion wherein the composition is forced through tubes in contact with the sheet while the sheet passes across the tube. A preferred technique comprises contacting the sheets with a head optionally equipped with a slit blade, i.e. a blade having cut-out areas, wherefrom the wax dispersion is extruded. By this technique the pattern, e.g. stripes, is defined by the design of the blade, which is inserted in the application head. The cut-out areas define the width of the stripes. Another application technique involves spraying or drippling the composition on a rotating surface such as a calender roll that then transfers the composition to the surface of the substrate.

Still another technique is based on traditional printing technologies, which comprise, for example, screen printing, roller printing and gravure printing. In general, printing comprises techniques wherein a rotating surface is provided with elevations (by engraving, embossing or similar techniques) and the elevations are contacted with the wax dispersion, e.g. by running it through a bath with liquefied phase one, and thus printed on the sheet. Whereas the slot coating technique preferably leads to patterns in machine direction as e.g. stripes, roller printing, screen printing or “Flexo coat” printing could be used to get a cross direction pattern.

Another technique to apply the wax dispersion is by using a screen printing procedure where the wax dispersion is introduced into a rotating roll and squeezed through a metal screen, which covers the roll. This leads, depending on the design of the screen, to a defined pattern on the fabric like stripes, dots, squares, circles and the like, or even logos and text.

A further technique to apply the wax dispersion onto the sheet is by roller-ball transfer, which comprises contacting a ball, which is in direct contact with the sheet, with wax dispersion in liquid state and transferring it through a rolling movement onto the sheet. Depending on the desired pattern of the wax dispersion on the sheet, there can be several of such roller-ball applicators mounted next to one another, or after one another. They may contain the same or different wax dispersions.

The wax dispersion may be applied by high-pressure coating. In one embodiment of this procedure the wax dispersion is applied via extrusion through appropriate nozzles, under high pressure. Specially shaped nozzles may be used resulting in particular patterns. For example there can be nozzles that result in circles, stars, squares, or other geometric shapes or even irregularly shaped patterns.

The wax dispersion may also be applied by a combination of these application techniques. The application of the wax phase on both sides can happen simultaneously or stepwise one side after the other.

In the instance of multi-layered sheet products, the wax dispersion may be applied to one or more of the sheet layers. This equally applies to the aqueous phase. Embodiments of multi-layered sheets are three-layered sheets wherein the outer layers have the same or different wax dispersions and the inner layers have an aqueous phase or any other combination.

The wax dispersion may be applied while being in mixture with water, which can be colored or uncolored. The added water or the water in the aqueous phase of the wax dispersion may be partially or completely removed after application to result in a semi-wet, a dry or essentially dry product. The water can be removed by evaporation which may be accomplished by a variety of means, e.g. by simply allowing the water to evaporate, by passing the sheet over one or more heated rolls, thus forcing the water to evaporate, by applying dry air, either heated or not, by applying reduced pressure. In the execution where the water is colored, it may diffuse into the sheet and after its evaporation leave the sheet colored. The wax dispersion that has been applied in this execution may be uncolored, in which case the wax particles will appear as white or lighter areas. Or the wax dispersion may be colored which will result in a multi-colored product. In another execution, the wax dispersion in this process is colored and uncolored water is used resulting in products wherein the wax dispersion areas are colored and the areas and the other areas are uncolored.

The thus obtained products may subsequently be treated with an aqueous phase which may be colored or not, resulting in products with even more color combinations.

The wax dispersion may also be applied as beads as described above. The wax dispersion can also be entrapped into dots, which are small bubbles of polymeric or other material that contain wax dispersion. In this instance it may be advantageous to make the wax dispersion more viscous. The dots may be introduced on the sheets by techniques similar as for applying stripes or other patterns, e.g. by contacting the sheet with a roll whereon the dots have been applied or a similar technique.

The wax dispersion preferably is applied in such manner that it will remain on the fabric surface or at the surface portion during the manufacturing process and storage. This can be conveniently accomplished by e.g. spraying or coating it to the surface of the sheet.

The wax dispersion preferably is applied such that it is present at the surface of the sheet because of its physical location in that instance, the wax dispersion is readily available to be spread onto the skin during usage. As a result, the effectiveness with which the wax dispersion is transferred to the skin during use, the availability and therefore the effectiveness of active ingredients is increased compared to products where the active agent is simply incorporated into a single continuously applied phase.

Additional Aqueous Phases

The products of the invention may contain, apart from one or more wax dispersions, one or more additional aqueous phases. These additional aqueous phases can be any of the art-known aqueous based formulations used to impregnate wipes. Beside water the additional aqueous phases may also contain further ingredients or additives such as surfactants, emulsifiers, consistency factors, conditioners, moisturizers, thickeners, preservatives, deodorants, film formers, powders, sunscreen filters, anti-oxidants, hydrotropes, active ingredients, in particular dermatologically active ingredients, fragrances and the like. Active ingredients as mentioned herein comprise, for example, anti-inflammatories, anti-bacterials, anti-fungals, biogenic actives and the like agents. Active ingredients suited for topical applications are particularly preferred.

The additional aqueous phases may contain suitable dyes that preferably are hydrophylic. In one type of embodiments, the wax dispersion is applied discontinuously as a layer e.g. in the form of stripes leaving areas with only aqueous phase, which areas are colored. This allows the manufacture of sheet products with colored patterns, e.g. colored lines or even multicolored patterns when the wax dispersion itself is also colored.

The additional aqueous phases may further contain lipophilic dyes, which upon contact with the wax dispersion migrate into that phase and cause it to become colored.

The additional aqueous phases may further contain one or more preservatives.

Suitable surfactants for the additional aqueous phases comprise:

alkyl sulfates, e.g. sodium lauryl sulfate, ammonium lauryl sulfate, sodium cetearyl sulfate;

alkyl sulfoacetates, e.g. sodium lauryl sulfoacetate;

alkyl ether sulfates e.g. sodium laureth sulfate, sodium trideceth sulfate, sodium oleth sulfate, ammonium laureth sulfate;

alkyl ether sulfosuccinates, e.g. disodium laureth sulfosuccinate;

alkyl glycosides, e.g. decyl glucoside, lauryl glucoside;

alkyl isothionates;

amphoterics, e.g. cocamidopropyl betaine, sodium cocoamphoacetate, sodium lauroamphoacetate, disodium lauroamphodiacetate, disodium cocoamphodiacetate, sodium lauroamphopropionate, disodium lauroamphodipropionate, potassium or ammonium salts of the aforementioned amphoterics, capryl/capramidopropyl betaine, undecyleneamidopropyl betaine, lauramidopropyl betaine and fatty alcohol polyglycol ethers.

Suitable conditioners are e.g. alkylamido ammonium lactate, cetrimonium chloride and distearoylethyl hydroxyethylmonium methosulfate and cetearyl alcohol, cetyl dimethicone, cetyl ricinoleate, dimethicone, laureth-23, laureth-4, polydecene, retinyl palmitate, agents selected from glyceryl monooleate and cocoglucoside including mixtures thereof (in particular the product ‘Lamesoft®’ of Cognis which is a mixture of these two components), quaternized protein hydrolysates, quaternized cellulose and starch derivatives, quaternized copolymers of acrylic or methacrylic acid or salts, quaternized silicone derivatives, silicone oils, cyclomethicones, and the like agents, including mixtures thereof.

Suitable thickeners are e.g. acrylates/steareth-20 methacrylate copolymer, carbomer, carboxymethyl starch, cera alba, dimethicone/vinyl dimethicone crosspolymer, propylene glycol alginate, hydroxyethylcellulose, hydroxypropyl methylcellulose, silica, silica dimethyl silylate, xanthan gum, hydrogenated butylene/ethylene/styrene copolymer.

The additional aqueous phases may further comprise film-forming substances lice chitosan and derivatives thereof, derivatives of poly acrylic acid, polyvinyl pyrrolidone and its derivatives, and the like.

The additional aqueous phases may contain pH sensitive components, i.e. components that change properties upon change of pH. The change of pH may occur when contacting the sheet product with the skin whereupon the pH changes from the pH of the product, which usually is about pH 7 to the skin pH which is about pH 5.5. pH sensitive agents for example comprise particular emulsifiers, stabilizers, surfactants viscosity regulating agents, chelators and the like.

In one embodiment an appropriate emulsifier is selected that is pH sensitive in this pH range in that it changes its emulsifying capacity, preferably increases its emulsifying capacity, so that upon contact with the skin an emulsification process occurs causing an interaction between the aqueous and wax phase in the wax dispersions.

The aforementioned change of pH that occurs upon application of the product to the skin may also promote the release from active ingredients, in particular actives that are pH sensitive, e.g. actives having a pH dependent solubility.

Application of the Additional Aqueous Phases

The additional aqueous phases may be applied to the sheet using methods generally known in the art for applying aqueous liquid lotions such as spraying, dripping, immersing and the like techniques. A preferred application method for the additional aqueous phases is by spraying with a suitable nozzle or by drippling, for example by using a perforated tube having holes or slits. The immersing technique can be done by running the sheets through a bath holding the additional aqueous phases and subsequently controlling the amount of liquid that is absorbed by pressing.

The additional aqueous phases may be applied in various ways as described for the lipid phase, evenly or non-evenly, continuously or non continuously, at the surface or surface portion or, preferably, throughout the whole of the sheet material. Optionally some parts of the sheet can be left dry, i.e. not having the lipid and the additional aqueous phases, or some parts can only have the lipid or the additional aqueous phases. The additional aqueous phases may be applied at both sides or only at one side of the sheet.

The additional aqueous phases can be applied in various amounts, for example in an amount from about 0.1 g to about 10 g per gram of substrate and is typically applied in an amount from about 1.0 g to about 10 g per gram of substrate, preferably from about 2.0 g to about 5 g per gram of substrate, most preferably from about 2 g to about 4.5 g per gram of dry substrate, most preferably about 3.7 to about 3.8 g per gram substrate. Or, the additional aqueous phases is applied in an amount of about 4 to about 8 g per wipe sized 17.2×21 cm, most preferably about 6 g per wipe. Or, alternatively, the additional aqueous phases are applied in an amount of about 3 to about 200 g/m², preferably of about 3 to about 40 g/m², more preferably from about 10 to about 20 g/m², either on one side or, preferably, on both sides of the sheet.

It may also be advantageous to only apply the additional aqueous phases to only those areas (or that side) of the sheet that have (or has) not already been covered with the wax dispersion.

Since in many cases the product is used as a cleansing article it is useful to design the additional aqueous phases as cleanser. Soils that are most difficult to clean are either water insoluble and/or strongly adhere to the skin. Therefore the additional aqueous phases are formulated such that it is capable of taking up water-insoluble materials.

Further Phases

In another embodiment of the invention a further layer is applied to the sheet, which is made of polymeric material, hereafter referred to as polymeric layer. The polymeric layer preferably is solid, i.e. having a melting point or melting range of above ambient temperature. One or more polymeric layers may be applied to the sheet. The term ‘polymeric layer’, whenever used hereinafter refers to one or more polymeric layers. The polymeric layer is made of a suitable polymer such as polyethylene, polypropylene, polyester, a silicone and the like, including mixtures thereof. The polymeric layer may contain other materials, such as fillers or dyes. In the latter instance the area of the sheet covered with the polymeric layer will occur as colored areas. In case several polymeric layers are applied, layers with different colors may be used thus resulting in different colored patterns.

The polymeric layer may be applied to the sheet similarly as described for the application of the wax phase. For example, the polymeric layer may be applied to one side of the sheet or to both sides, it may be applied continuously, i.e. over the whole surface of the sheet, or discontinuously, e.g. in patterns, e.g. as stripes, spots or other figures. In the instance where the polymeric layer does not cover the whole surface, the wax phase may cover both the areas of the sheet that are covered by the polymeric layer and the other areas.

The wax dispersion layer may be applied onto the polymeric layer thus forming a double layer. The polymeric layer needs not be completely covered by the wax phase, i.e. some parts may remain uncovered.

The polymeric layer may also be applied to the areas that are not covered by the wax dispersion. For example the wax dispersion may be applied as a layer in a discontinuous fashion and the polymeric phase is applied at the spots without wax dispersion. In one particular embodiment the wax dispersion is applied as stripes and the polymeric layer is put in the area between these stripes thus forming a pattern of alternating stripes of wax dispersion and polymeric layer. This may for example be done at one side of the sheet while the aqueous phase is put at the other side.

The polymeric layer may be semi-solid so that it can be disrupted upon application of a product having such a layer. Semi-solid polymeric layers are made of polymers that have a waxy, creamy or similar consistency. In that instance the polymeric layer can also be applied as an external coating onto the sheet, covering one or both sides, covering parts or the whole surface. It may also cover parts or the whole of the wax dispersion layer.

The wax dispersion that covers the polymeric layer may be colored or uncolored. In the former instance, the polymeric layer preferably is uncolored or white although it may be colored also. In the instance where the wax dispersion is uncolored, the polymeric phase preferably is colored, although it may also be white or uncolored.

The polymeric phase may be applied for improving or promoting the transfer of the wax dispersion that is coated thereon to the user's skin. Using a colored polymeric layer, or a colored wax dispersion, or both, results in an appearance, disappearance or respectively change of color when the sheet product is used and the wax dispersion is transferred to the skin.

The polymeric layer is applied to the sheet using art-known methods to coat sheet-like materials with a polymeric layer. For example the polymeric layer can be applied by screen printing, gravure printing, roller printing, embossing, spraying, drippling, bathing and the like techniques.

In some embodiments of this invention the products may contain two or more wax dispersions with different stability towards the aqueous phase. This allows one phase to interact more quickly with the aqueous phase than the other. This may find application in products where a gradual release of active ingredient is desired or the release of a sequence of two or more active ingredients.

Manufacture

This invention further concerns a process for preparing a product as specified herein, said process comprising contacting a porous or absorbent sheet with a wax dispersion, and if desired with further wax dispersions, said wax dispersion or further wax dispersions being as described herein.

In another aspect, the invention concerns a process for preparing a product as specified herein, said process comprising contacting a porous or absorbent sheet with a wax dispersion and an additional aqueous phase, both being as described herein, or if desired with further wax dispersions and/or with further additional aqueous phases. The porous or absorbent sheet in particular is made of a non-woven material. In another aspect, the invention concerns the process of contacting the sheet simultaneously or subsequently with the wax dispersion and the aqueous phase. In a particular execution, the process comprises contacting the sheet with a wax dispersion and subsequently with an additional aqueous phase.

The wax dispersion and additional aqueous phase can be applied to the sheet at any time during the manufacturing process of the sheet, for example either one or both of the phases may be applied during the manufacturing process of the sheet material. Preferably the wax dispersion and/or additional aqueous phase can be applied to the sheet after finishing the manufacturing process of the sheet, more preferably after the wax dispersion and/or aqueous phase can be applied to the sheet after finishing the manufacturing process of the sheet, more preferably after the sheet has been dried.

In a particular execution, the sheet material is cut into strips, the transversal size of which being similar to the size of the tissue, wipe or towelette. Subsequently, the wax dispersion and where desired, the additional aqueous phase, are applied to these strips. Where both types of phases are applied, preferably first the wax dispersion is applied and then the additional aqueous phase. Thereafter the strips are folded according to methods generally known and applied in the art. In an alternative execution, the wax dispersion is applied to these strips, which are subsequently folded. If desired, the thus folded strips may be moistened with an additional aqueous phase as, said moistening preferably comprising spraying or drippling, or by immersing in or running the strip through a bath containing the additional aqueous phase. The strips can be dried after application of the aqueous phase using methods described hereinabove.

In a further step, the strips are cut so that the desired size of the sheets, in particular of

[symbol]

Although in preferred executions the wax dispersion is not present on the whole surface of the wipe, good release of the wax dispersion and of the components contained therein is attained, in particular when the local emulsification process comes into play.

Optimal release of active ingredients can also be achieved by making use of both above possibilities.

The products according to the invention can take the form of baby or adult wipes and can be used in a wide range of applications as personal care products, comprising, for example, baby cleansing wipes, face or body cleansing wipes, wipes for skin treatment or skin conditioning such as for example skin moisturization and against skin ageing, insect repellent wipes, powder wipes, toilet wipes, antiperspirant wipes, peeling wipes, after-sun treatment wipes, sunscreen wipes, wipes for feminine hygiene, nappy rash wipes, the latter preferably containing zinc oxide as active ingredient, and the like.

The products of the invention, which are dry or essentially dry, are mainly aimed for use as end products. In this instance the consumer is instructed to treat these products with water or with an aqueous lotion, which for example could be sold separately.

Examples of products with low water content are the so-called ‘dry wipes’, which are aimed for use on a wet skin. Examples of applications for this type of wipes are usage in the shower or after bathing. Such dry wipes may also be recommended for use after wetting the product itself, e.g. with water or with an aqueous lotion that is provided separately. Examples of products with relatively higher water content from about 5% to about 10% are so-called ‘intermediate dry wipes’, which may find application as inserts in diapers.

The products of the invention may also be used as cleansing tools. They have been found to be more effective cleansers compared to products that have only an aqueous phase. This is due, i.a., to the fact that they are effective in removing both aqueous and lipid soils.

The products of the invention can also be used as applicators of active substances or other ingredients having skin-beneficial properties (i.e. the so-called ‘leave-on’ ingredients), in particular of any of the active substances mentioned herein, or they find use as combined cleanser and applicator of active substances. Furthermore, the described products deliver a better and long-lasting protection of the skin by an effective release of skin-protecting ingredients to the skin.

So far, wipes have been used primarily as a cleansing tool. Applications as a vehicle for active substances or any other leave-on products, have been limited because of the poor transfer rate of the active ingredients from the wipe to the skin. The products of this invention have the advantage of having an excellent transfer of active ingredients to the skin thus widening the applications of wipe products as a vehicle for a number of actives, in particular more expensive actives that so far could not be applied through wipes because of their poor transfer rate. The products of this invention additionally provide other consumer benefits such as a more even distribution of the actives on the skin and a better skin penetration.

Either of both aspects may be present to a larger extent, i.e. the product may be primarily for cleansing purposes but also have the capability of transferring certain beneficial components or active substances to the skin, or vice versa, the products may be designed for applications in instances where the primary benefit is not cleansing but a better and more convenient form of application of leave-on products. Hence the products of this invention allow the possibility of being optimized towards two key consumer needs—cleansing and caring.

Another benefit of the products of this invention is that they offer a softer feel of the fabric due to the modification of the fabric surface caused by the presence of the wax dispersion. The products moreover offer gentler cleansing because of less friction of the wipe on the skin (softer skin-feel). As used herein, softness refers to the tactile sensation perceived when the consumer holds the product, rubs it across the skin, or crumples it with the hand.

The products of this invention additionally offer the possibility to incorporate into or apply to one product two or more incompatible ingredients, thus allowing the user to apply incompatible agents with one and the same product. In particular it is possible to have a product that has as well water soluble as lipid soluble ingredients, for example a wipe that has active ingredients that are water-soluble and oil soluble.

A still further advantage of those products where the wax dispersion is at the surface lies in the fact that the instant products allow an improved transfer of actives onto the skin since the active ingredients are concentrated on the surface of the sheet/fabric material and are not included in the inner phase of a typical o/w-emulsion.

The products according to the invention possess the additional advantage of being almost odorless (unless fragrances are added), environmentally friendly and biologically decomposable.

In view of these beneficial properties, the products of this invention can be used in a wide variety of cosmetic and personal care applications, but also in other cleaning or cleansing applications such as cleaning of hard surfaces (household care).

### EXAMPLES

The following examples are given with the nomenclature of INCI. As used in the following examples, C.I. refers to dyes.

### Examples 1-11

Manufacturing procedure of the wax dispersion in a 1000 kg mixer.

Distearyl carbonate (20 kg), distearyl ether (20 kg), lauryl alcohol C12 (50 kg), myristyl alcohol 14 (20 kg), cetyl alcohol (20 kg), stearyl alcohol (10 kg), paraffinum perliquidum DAB (20 kg), a part of the emulsifier ceteareth-20 (3,0 kg), polyglyceryl-2 dipolyhydroxy stereate (1.0 kg), a part of the polymer polyacryl-amide C₁₃-C₁₄ iso paraffin laureth-7 (1.0 kg) and a part of dist. water (430 kg) were mixed in a heat [symbol] during the addition of the preemulsion. Subsequently, phenoxyethanol (5.0 kg) was added under stirring as to preserve the wax dispersion.

The viscosity of the wax dispersion measured with Brookfield RVF, spindle 5, 10 rpm was 30.000 mPa·s (at 23° C.). The particle size determination was done by Fraunhofer-deflection (Mastersizer 2000, Malvern Instruments Ltd.) and resulted in a particle size range d(0.5) of 10 μm and d(0.9) of 30 micrometers, meaning that 50% of the dispersed material had a particle size distribution smaller than 10 μm and 90% smaller than 30 μm.

The amounts in the following examples are given in w/w-%. For the commercially available substances the w/w-% is based on the weight of the substance as sold, including solvents and other ingredients. The following table lists examples of dispersions made in accordance with the above methodology.

### Example List 1-11

### Wax Dispersions

| | Ex. 1 | Ex. 2 | Ex. 3 | Ex. 4 | Ex. 5 | Ex. 6 |
|---|---|---|---|---|---|---|
| | w/w | w/w | w/w | w/w | w/w | w/w |
| INCI | % | % | % | % | % | % |
| Distearyl | 3.0 | — | 2.5 | — | 1.0 | — |
| Carbonate | | | | | | |
| Distearyl Ether | — | 2.0 | 2.5 | — | — | — |
| Dibehenyl Ether | — | 1.0 | — | — | — | — |
| Azelainic acid | 2.0 | — | — | 1.0 | 1.0 | — |
| 12-Hydroxystearyl- | — | 4.0 | — | 2.5 | — | — |
| alkohol | | | | | | |
| Lauryl Alcohol | 3.0 | 2.0 | 3.0 | 4.0 | 1.0 | — |

| | Ex. 7 | Ex. 8 | Ex. 9 | Ex. 10 | Ex. 11 |
|---|---|---|---|---|---|
| | w/w | w/w | w/w | w/w | w/w |
| INCI | % | % | % | % | % |
| Distearyl Carbonate | — | — | — | — | — |
| Distearyl Ether | 1.0 | — | 3.0 | 0.2 | 0.2 |
| Dibehenyl Ether | — | — | — | — | — |
| Azelainic acid | — | — | — | — | — |
| 12-Hydroxystearyl- | — | — | — | — | — |
| alkohol | | | | | |
| Lauryl Alcohol | — | — | — | — | — |
| Myristyl Alcohol | 6.0 | — | — | — | — |
| Cetyl Alcohol | — | — | — | — | — |
| Stearyl Alcohol | 3.0 | 6.6 | — | 3.0 | — |
| Cetearyl Alcohol | — | — | — | — | — |
| Glyceryl Stearate | 2.0 | — | 5.0 | 5.0 | 5.0 |
| Glycol Distearate | — | — | — | — | — |
| Cocoglycerides | 5.0 | 13.0 | 9.0 | 8.8 | 11.8 |
| Hydrogenated | — | — | — | — | — |
| Vegetable Oil | | | | | |
| Shorea Stenoptera | — | — | — | — | — |
| Cetearyl Glucoside | — | — | — | — | — |
| (and) Cetearyl | | | | | |
| Alcohol | | | | | |
| Lauryl Glucoside | — | — | — | — | — |
| (and) Polyglyceryl-2 | | | | | |
| Dipolyhydroxystearate | | | | | |
| (and) Glycerin | | | | | |
| Ceteareth-12 | — | — | — | — | — |
| Ceteareth-20 | — | — | — | — | — |
| Sodium Cetearyl | — | — | — | — | — |
| Sulfate | | | | | |
| Polyglyceryl-2 | — | — | — | — | — |
| Dipolyhydroxy- | | | | | |
| stearate | | | | | |
| Octyl Stearate | — | — | — | — | — |
| Dicaprylyl Ether | — | — | — | — | — |
| Paraffinum liquidum | — | — | — | — | — |
| Olus (EU), | — | — | — | — | — |
| Vegetable Oil (non | | | | | |
| EU) | | | | | |
| Dicaprylyl | — | — | — | — | — |
| Carbonate | | | | | |
| Cocoglycerides | — | — | — | — | — |
| Hydroxyethyl- | — | — | — | — | — |
| cellulose | | | | | |
| Glycerin (and) | — | — | — | — | — |
| Glyceryl | | | | | |
| Polyacrylate | | | | | |
| Hydroxypropyl Guar | — | — | — | — | — |
| Aqua (and) Sodium | — | — | — | — | — |
| Polyacrylate | | | | | |
| C13-Cl4 Isoparaffin | — | — | — | — | — |
| (and) Laureth-7 | | | | | |
| (and) | | | | | |
| Polyacrylamide | | | | | |
| PEG-20 | — | — | — | — | — |
| Monostearate | | | | | |
| Panthenol | — | — | — | — | — |
| Butylene Glycol | — | — | — | — | — |
| Glycerin | — | — | — | — | — |
| Bisabolol | — | 0.3 | — | — | — |
| Glycine Soja | — | — | — | — | — |
| Glyceryl Laurate | 3.0 | — | 3.0 | 3.0 | 3.0 |
| PEG-40 | 4.0 | 4.0 | 4.0 | 4.0 | 4.0 |
| Hydrogenated | | | | | |
| Castor Oil | | | | | |
| Colorant | 0.003 | 0.003 | 0.003 | 1.4 | 0.7 |
| Preservative | qs | qs | qs | qs | qs |
| Aqua | Ad 100 | Ad 100 | Ad 100 | Ad 100 | Ad 100 |

### Example 12

### Aqueous Phases

| Phase 12-A | |
|---|---|
| Aqua | 96.336% |
| Polysorbate 20 | 0.600% |
| PEG-75 Lanolin | 0.100% |
| Parfum | 0.150% |
| PEG-40 Hydrogenated Castor Oil | 0.400% |
| Propylene Glycol | 1.120% |
| Phenoxyethanol | 0.800% |
| Tetrasodium EDTA | 0.078% |
| Chamomilla Recutita | 0.070% |
| Ethoxydiglycol | 0.171% |
| Butylene Glycol | 0.035% |
| Glucose | 0.016% |
| Iodopropynyl Butylcarbamate | 0.010% |
| PEG-4 Laurate | 0.090% |
| Citric Acid | 0.020% |
| Phase 12-B | |
| Aqua | 98.252% |
| Phenoxyethanol | 0.800% |
| Iodopropynyl Butylcarbamate | 0.010% |
| PEG-4 Laurate | 0.090% |
| Parfum | 0.150% |
| Tetrasodium EDTA | 0.078% |
| Citric Acid | 0.020% |
| Polysorbate 20 | 0.600% |
| Phase 12-C | |
| Aqua | 97.250% |
| Glycerines | 1.000% |
| Phenoxyethanol | 0.800% |
| Iodopropynyl Butylcarbamate | 0.010% |
| PEG-4 Laurate | 0.090% |
| Parfum | 0.150% |
| Tetrasodium EDTA | 0.078% |
| Citric Acid | 0.020% |
| Polysorbate 20 | 0.600% |
| Phase 12-D | |
| Aqua | 96.332% |
| Glycerines | 1.000% |
| Phenoxyethanol | 0.800% |
| Polysorbate 20 | 0.600% |
| PPG-15 Stearyl Ether | 0.400% |
| PEG-7 Glyceryl Cocoate | 0.100% |
| Propylene Glycol | 0.350% |
| Iodopropynyl Butylcarbamate | 0.010% |
| PEG-4 Laurate | 0.090% |
| Chamomilla Recutita | 0.070% |
| Parfum | 0.150% |
| Tetrasodium EDTA | 0.078% |
| Citric Acid | 0.020% |
| Phase 12-E | |
| Aqua | 97.33% |
| Phenoxyethanol | 0.800% |
| Polysorbate 20 | 0.600% |
| Sorbeth-30 | 0.400% |
| Propylene Glycol | 0.350% |
| Dimethicone Copolyol | 0.100% |
| Iodopropynyl Butylcarbamate | 0.010% |
| PEG- 4 Laurate | 0.090% |
| Chamomilla Recutita | 0.070% |
| Parfum | 0.150% |
| Tetrasodium EDTA | 0.078% |
| Citric Acid | 0.020% |
| Phase 12-F | |
| Aqua | 97.332% |
| Phenoxyethanol | 0.800% |
| PEG-80 Sorbitan Laurate | 0.600% |
| Propylene Glycol | 0.350% |
| Sorbeth-30 | 0.400% |
| Octyldecanol | 0.100% |
| Iodopropynyl Butylcarbamate | 0.010% |
| PEG-4 Laurate | 0.090% |
| Chamomilla Recutita | 0.070% |
| Parfum | 0.150% |
| Tetrasodium EDTA | 0.078% |
| Citric Acid | 0.020% |
| Phase 12-G | |
| Aqua | 97.332% |
| Phenoxyethanol | 0.800% |
| Polysorbate-20 | 0.600% |
| PGG-15 Stearyl Ether | 0.400% |
| Propylene Glycol | 0.350% |
| Decyl Oleate | 0.100% |
| Iodopropynyl Butylcarbamate | 0.010% |
| PEG-4 Laurate | 0.090% |
| Chamomilla Recutita | 0.070% |
| Parfum | 0.150% |
| Tetrasodium EDTA | 0.078% |
| Citric Acid | 0.020% |
| Phase 12-H | |
| Sodium Myreth Sulfate | 10.00% |
| Lauryl Glucoside | 15.00% |
| Cocamidopropyl Betaine | 10.00% |
| Aqua | 64.50% |
| Parfum | 0.50% |
| Phase 12-I | |
| Sodium Laureth Sulfate | 20.00% |
| Decyl Glucoside | 5.00% |
| Cocamidopropyl Betaine | 8.00% |
| Laureth-2 | 2.50% |
| Polysorbate-20 | 1.00% |
| Aqua | 63.00% |
| Parfum | 0.50% |
| Phase 12-J | |
| Sodium Myreth Sulfate | 15.00% |
| Lauryl Glucoside | 10.00% |
| Laureth-2 | 1.50% |
| Aqua | 73.00% |
| Parfum | 0.50% |
| Phase 12-K | |
| Emulgade ® CM | 20.00% |
| Polysorbate 20 | 0.80% |
| Coco-Glucoside | 2.50% |
| Phenoxyethanol | 1.00% |
| Cetylpyridinium Cloride | 0.10% |
| Tetrasodium EDTA | 0.20% |
| Aqua | 75.22% |
| Citric Acid | 0.08% |
| Parfum | 0.10% |
| Phase 12-L | |
| Emulgade ® SE-PF | 1.66% |
| Ceteareth-12 | 0.94% |
| Lamesoft ® PO 65 | 0.25% |
| Paraffinum Liquidum | 3.00% |
| Cetylpyridinium Cloride | 0.05% |
| Polysorbate-20 | 1.00% |
| Citric Acid | 0.03% |
| Tetrasodium EDTA | 0.20% |
| Nipaguard ® IPF | 0.10% |
| Aqua | 92.66% |
| Parfum | 0.11% |
| Phase 12-M | |
| Propylene Glycol | 0.98% |
| PEG-40 Hydrogenated Castor Oil | 0.4% |
| Butylparaben | 0.075% |
| Methylparaben | 0.155% |
| Propylparaben | 0.1% |
| Phenoxyethanol | 0.7% |
| Polysorbate-20 | 3.25% |
| Citric Acid | 0.05% |
| Tetrasodium EDTA | 0.2% |
| Nipaguard ® IPF | 0.098% |
| Aqua | 93.94% |
| Parfum | 0.15% |

### Example 13

Dry hydro-entangled sheet material made of fabric having a surface weight of 50 g/m2 was cut into strips. The wax dispersion, prepared as set forth in example list 1-12, was applied onto both sides of the fabric with the total amount of 1.0 g in the form of a thin layer by using the slot coating process. This comprised running the fabric strips against two heads each mounted on one side of the fabric strip. Subsequently the strips were folded, cut and formed to stacks, which were packed into laminate.

### Example 14

Strips treated with a wax dispersion were prepared as described in the previous example. The strips were subsequently sprayed in the conventional manner with the liquid as prepared in example list 12. Liquid addition was set at 6 g per wipe. Subsequently the strips were folded, cut and formed to stacks, which were packed into laminate.

### Example 15

Strips treated with a wax dispersion were prepared as described in the previous example. The strips were subsequently dried by blowing dry air (no heating) over the fabric strips. Subsequently the strips were folded, cut and formed to stacks, which were packed into laminate.

1. A product comprising a porous or absorbent sheet whereto a wax dispersion has been applied.

2. A product according to claim 1 wherein the wax dispersion comprises
(a) a wax phase comprising suitable wax components, said wax phase having a melting point or melting range which is above ambient temperature, or which in particular is equal or higher than 25° C., and
(b) an aqueous phase.

3. A product according to claim 2 wherein the wax phase mentioned under (a) is present in an amount of 1-75 w/w-% and the aqueous phase is present in an amount of 25-99 w/w-%, relative to the total weight of the dispersion.

4. A product according to claim 3 wherein the wax dispersion further contains a suitable emulsifier.

5. A product according to claim 1 wherein the wax dispersion comprises wax particles that comprise a wax phase containing components selected from fats, waxes, fatty alcohols, fatty acids, dialkyl(ene) ethers, dialkyl(ene) carbonates, dicarbonic acids and hydroxyfatty alcohols, and any mixture of these components.

6. A product according to claim 1 wherein the wax phase comprises mono-, di- or triglycerides, fatty alcohols, fatty acids or any combination of these ingredients.

7. A product according to claim 5 wherein the wax phase contains components selected from dialkyl(ene) ethers or -carbonates, dicarboxylic acids or hydroxy fatty alcohols and any combination thereof.

8. A product according to claims 1 wherein the wax particles have an average particle size, which is in the range of 0.5 to 100 μm.

9. A product according to claim 1 wherein the wax dispersion comprises
(a) a wax phase having a melting point which is equal or higher than 25° C., that contains at least one oil or wax component selected from dialkyl(ene) ethers, dialkyl(ene) carbonates, dicarboxylic acids or hydroxyfatty alcohols or any mixture of these substances and at least contains one emulsifier, and
(b) an aqueous phase.

10. A product according to claim 9 wherein the wax dispersion contains
(a) 1-75 w/w-% of a wax phase having a melting point higher than 25° C., that contains at least one oil or wax component selected from dialkyl(ene) ethers, dialkyl(ene) carbonates, dicarbonic acids or hydroxyfatty alcohols or any mixture of these substances and contains at least one emulsifier, and
(b) 25-99 w/w-% of an aqueous phase, relative to the total weight of the complete composition.

11. A product according to claim 1 wherein the wax dispersion comprises:
(a) 1-50 weight % of a wax phase comprising:
(a1) 0.1-30 weight % of at least an oil or wax component selected from C₁₄-C₃₀-dialkyl ethers, C₁₄-C₃₀-dialkyl carbonates, C₄-C₃₄-dicarbonic acids or C₁₂-C₃₀-hydroxyfatty alcohols or any mixture thereof;
(a2) 0.1-10% (w/w) of at least one oil;
(a3) 0.1-10% (w/w) of at least one non-ionic emulsifier
(a4) 0.1-40% (w/w) of at least one further waxy lipid component; w/w relative to the total weight of the wax dispersion;
(b) 50-99% (w/w) of an aqueous phase; w/w relative to the total weight of the wax dispersion.

12. A product according to claim 1 wherein the wax dispersion contains 5-30 weight % of the wax phase relative to the total weight of the wax dispersion.

13. A product according to claim 1 wherein the wax dispersion additionally contains a 0.01-5% (w/w relative to the total weight of the dispersion) of a polymer.

14. A product according to claim 13, wherein the polymer is selected from the group of polyacrylates, polysaccharides, polyacrylamides or any mixture of these polymers.

15. A product according to claim 1 wherein an additional aqueous phase has been applied to the sheet.

16-23. (canceled)

24. A method of manufacturing a product as claimed in claim 1, said method comprising applying to the sheet a wax dispersion.

26. A method of manufacturing a product as in claim 15, said method comprising applying to the sheet a wax dispersion and a drying step.

27-29. (canceled)
